(19) United States
(12) Patent Application Publication
(10) Pub. No.: US 2026/0354783 A1
(43) Pub. Date: Nov. 12, 2026

(54) Artificial Intelligence for Automated Calcium Quantification From Non-Contrast Enhanced Images in Patients With Degenerative Aortic Stenosis

(71) Applicants: The MITRE Corporation, McLean, VA (US); University of Ottawa Heart Institute, Ottawa, Ontario (CA)

(72) Inventors: Andy Taylor, Amherst, NY (US); Jocelyn Ornelas Munoz, Merced, CA (US); David Michael Slater, Herndon, VA (US); Pascal Theriault Lauzier, Ottawa, Ontario (CA); David Messika-Zeitou, Ottawa, Ontario (CA)

(21) Appl. No.: 19/124,772

(22) PCT Filed: Oct. 27, 2023
(86) PCT No.: PCT/CA2023/051436

§ 371 Date: Apr. 25, 2025

Related U.S. Application Data
(60) Provisional application No. 63/420,270, filed on Oct. 28, 2022.

(51) Int. Cl. A61B 6/00; A61B 6/03; A61B 6/50; G06T 7/00; G06T 7/73; G16H 50/20

(52) U.S. Cl. CPC A61B 6/5217; A61B 6/032; A61B 6/503; G06T 7/0012; G06T 7/73; G16H 50/20; G06T 2207/10081; G06T 2207/20081; G06T 2207/20084; G06T 2207/30048; G06T 2207/30101

(57) ABSTRACT

The present invention is directed to artificial intelligence base deep learning methods to automate the quantification of aortic root calcification using non-contrast computed tomography (CT) scan images. The computer implemented methods employ a multi-level convolutional neural network to localize the 3D position and/or the 3D orientation of an isotropic centroid of the aortic valve on the CT image. The 3D position and/or the 3D orientation of the centroid is employed by a segmentation-algorithm, a deep learning convoluted neural network, to segment an area surrounding the isotropic centroid into voxels and quantify the number of voxels containing calcium to calculate an Aortic Valve Calcification score, which can be used to determine the severity of Aortic Stenosis in a patient.

### INCORPORATION BY REFERENCE

All publications, patents and patent applications referred to herein are incorporated by reference in their entirety to the same extent as if each individual publication, patent or patent application was specifically and individually indicated to be incorporated by reference in its entirety.

### FIELD OF THE INVENTION

This invention relates to a computer-implemented method of locating an isotropic centroid of an aortic root on a non-contrast image to quantify calcification of the aortic root to determine severity of aortic stenosis and other cardiac conditions. More specifically, the invention relates to deep learning based convolutional neural networks to automate the quantification of aortic valve calcification.

### BACKGROUND

There has been significant effort developed in the past few years to use deep learning or machine learning models as well as convolutional neural networks for automatic quantification of cardiac calcification measured using CT but most of the literature has focused on the quantification of coronary calcium (CAC), a well-established predictor of cardiovascular risk. Similarly to AVC, CAC scoring requires excluding non-coronary calcium from the measurement, but challenges are markedly different due to anatomical considerations. CAC extends into several small lesions distributed over a large volume and CAC is usually well isolated from high-attenuation structures due to the epicardial course of coronary arteries. In contrast, AVC is localized within a much smaller volume and often in continuity with calcium in the walls of the aorta, coronary arteries and the mitral annulus.

Aortic valve calcification (AVC) is the main pathophysiological process leading to degenerative aortic stenosis (AS). AVC is the most common cause of aortic valve stenosis (AS), a condition with high mortality and morbidity once it becomes severe. AVC can be accurately and quantitively assessed using non-contrast ECG-gated computed tomography (CT). The degree of AVC is closely related to AS hemodynamic severity and is currently recommended for the assessment of AS severity in patients with low flow and preserved or reduced ejection fraction), as well as in cases when echocardiographic assessment is discordant. The AVC score is measured using a dedicated software, which identifies areas of calcification and allows the operator to manually segment regions deemed to be part of the aortic valve. AVC measurement requires training and operator expertise and usually takes several minutes per scan.

CT AVC scoring is currently recommended as a complementary method for the evaluation of AS severity in clinical situations such as low-flow low-gradient AS with either preserved or reduced left ventricular ejection fraction. Thresholds for AS severity have been well validated and are now implemented both in European and North American clinical guidelines.

Conventionally, manual segmentation of contrast and non-contrast cardiac computed tomography (CT) scan images have been used to quantify the amount of calcification and determine AVC scoring which is a relatively time-consuming and repetitive task. AVC scoring on non-contrast gated CT is the guideline recommended quantification method and in clinical practice, the segmentation of AVC from non-AVC calcium is performed manually. These clinically available methods for calcium quantification involving manual selection of regions of calcification that are part of the aortic valve are cumbersome, time consuming, and prone to error.

### SUMMARY OF THE INVENTION

In an embodiment of the invention, a computer-implemented method of locating an isotropic centroid of an aortic root is provided. The method involves providing a non-contrast cardiac computed tomography image (CT image) to a multi-level convolutional neural network (ML-CNN). The CT image is processed by the ML-CNN to identify a 3D position and/or a 3D orientation of a plurality of anatomical features of the aortic root. The 3D position and/or 3D orientation of the anatomical features allows prediction of a 3D position and/or 3D orientation of the isotropic centroid of the aortic root on the CT image.

In a further embodiment, the ML-CNN predicts the 3D position and/or 3D orientation of the isotropic centroid by processing/surveying the CT image to first identify the image 3D midpoint. This is followed by refining the spatial resolution and restricting the field of view to focus on the area surrounding the 3D midpoint of the CT image with an isotropic voxel spacing that captures the entire aortic root. It is pertinent to note that the image matrix is kept constant and remain unchanged unlike the resolution and field of view. Once the ML-CNN is focused on the area surrounding the 3D midpoint, the 3D position and/or 3D orientation of the plurality of anatomical features of the aortic root are identified which allows prediction of the 3D position and/or 3D orientation of the isotropic centroid of the aortic root.

In some embodiments, the steps of identifying the 3D position and/or 3D orientation of the plurality of anatomical features of the aortic root and its isotropic centroid are repeated multiple times by the ML-CNN by further refining the spatial resolution and restricting the field of view each time while keeping the image matrix constant. After every prediction, the ML-CNN uses a previous prediction of the 3D position and/or 3D orientation of the isotropic centroid of the aortic root as a starting point before predicting a new 3D position and/or 3D orientation of the isotropic centroid.

The plurality of anatomical features that could be identified by the ML-CNN can be an aortic valve annulus centroid, at least one aortic valve commissure, an aortic leaflet coaptation point, and at least one coronary ostia.

In some embodiments, the plurality of anatomical features are identified by determining the axial distance between each anatomical feature. In some further embodiments, the plurality of anatomical features are identified as geometric points on the CT image by using a different colour for each anatomical feature.

In some embodiments, the ML-CNN restricts the field of view to an isotropic voxel spacing of 4 mm, or 2 mm, or 1 mm. In some embodiments, the field of view could be restricted to any isotropic voxel spacing as long as the image matrix is kept constant. The ML-CNN can maintain the CT image size matrix to 64 cube voxel, 32 cube voxel, 24 cube voxel, 16 cube voxel, 8 cube voxel or 1 cube voxel.

In some embodiments, the ML-CNN may predict the isotropic centroid of each aortic leaflet separately. In some embodiments, the ML-CNN may predict an edge of the left cusp, right cusp, non-coronary cusp and left ventricular outflow tract [LVOT] regions on the CT image.

In some further embodiments, the computer-implemented method and the ML-CNN algorithm may involve use a cardiac image generated using a non-CT medical imaging technique. In such embodiments, ML-CNN processes the cardiac image to predict the 3D position and/or 3D orientation of the isotropic centroid of the aortic root.

In some embodiments, the ML-CNN may be trained to accurately identify 3D position and/or 3D orientation of the plurality of anatomical features of the aortic root and to accurately predict the 3D position and/or 3D orientation of the isotropic centroid of the aortic root based on the 3D position and/or 3D orientation of the anatomical features. The training may involve manually annotating a plurality of CT images and determining the 3D position and/or 3D orientation of the isotropic centroid of the aortic root to generate a training module. Once the manual annotations are complete, the ML-CNN can be trained using the training module for a plurality of epochs. In some embodiments, the training may require altering the orientation of the plurality of CT images and allowing the ML-CNN to determine the isotropic centroid of the aortic root after the CT images are altered. Once the 3D position and/or 3D orientation of the isotropic centroid of the aortic root is predicted by the ML-CNN, it can be compared with the 3D position and/or 3D orientation that was predicted during manual annotations. The alteration of CT images may include random alteration of the orientation of the plurality of images by varying the image field of view, by rotating the image or by scaling-down pixel spacing of the image. The training may also include testing the ML-CNN by providing a new CT image to the ML-CNN for predicting the 3D position and/or 3D orientation of the isotropic centroid of the aortic root, wherein the isotropic centroid of the aortic root on the CT image is already known/has been identified previously by manual annotation. Thereafter, accuracy of the ML-CNN's prediction can be determined by comparing the 3D position and/or 3D orientation of the isotropic centroid predicted by the ML-CNN with the 3D position and/or 3D orientation identified previously. In some embodiments, the accuracy of the ML-CNN is validated using a nine-fold cross validation technique by determining a 3D position and/or 3D orientation error and/or a 3D angular error.

In some embodiments, the method is used to locate 3D position and/or 3D orientation of an isotropic centroid of any organ or part thereof on a medical image and the 3D position and/or 3D orientation of the isotropic centroid of the organ or part thereof could be useful for surgical planning.

In some embodiments, a computer-implemented method of quantifying an amount of calcium in an aortic root is provided. The method involves providing a non-contrast cardiac computed tomography image (CT image) to a deep-learning convolutional neural Network (DL-CNN), wherein 3D position and/or 3D orientation of an isotropic centroid of the aortic root on the CT image is known/has been identified previously. The CT image is then processed to quantify the amount of calcium using the DL-CNN, where the DL-CNN segments a pre-determined region surrounding the isotropic centroid into a plurality of voxels; surveys each segmented voxel to ascertain whether it contains calcium; and calculates an Aortic Valve Calcification (AVC) score by quantifying the number of calcium containing segmented voxels that form part of the aortic root.

In some embodiments, the CT image provided to the DL-CNN has a field of view defined by n×512×512, where n is the number of CT slices. The DL-CNN restricts the field of view to 32×128×128, 16×64×64, 8×32×32, 4×16×16 or 2×4×4 prior to segmenting the pre-determined region to capture the entire aortic root on the CT image.

In some embodiments, the DL-CNN assigns a score of 1 to a segmented voxel that contains calcium and a score of 0 to a segmented voxel that does not contain calcium. It is pertinent to note that DL-CNN does not take into account a segmented voxel that contains calcium but does not form part of the aortic valve. DL-CNN identifies the segmented voxel containing calcium by determining a CT attenuation value and a CT attenuation value of ≥130 HU indicates that the segmented voxel contains calcium.

In some embodiments, the DL-CNN creates a mask that displays the segmented voxels that were identified as containing calcium. In some further embodiments, the DL-CNN quantifies the amount of calcium of each aortic leaflet separately. In some further embodiments, the DL-CNN quantifies the amount of calcium of left cusp, right cusp, non-coronary cusp and left ventricular outflow tract [LVOT] separately.

The AVC score assigned by DL-CNN is an indicator or various medical conditions. For instance, a higher AVC score is an indicator of cardiac conditions, or an indicator of Aortic Stenosis. In some embodiments, the proposed segmentation and quantification method can be carried out to track the progress of Aortic Stenosis. A higher AVC score could also be an indicator of periprocedural complications in patients undergoing transcatheter aortic valve implantation (TAVI). The presence of calcium/a higher amount of calcium in the LVOT is an indicator of need for pacemaker implantation or paravalvular leak after TAVI. In some embodiment, the method may be employed to quantify the amount of calcium in any organ or a part thereof on a medical image.

In some embodiments, the DL-CNN may be composed of more than one neural network models. For instance, the DL-CNN could be a combination of two or more neural network models e.g. the DL-CNN could be a combination of a U-Net model and a ResNet model.

In some further embodiments, the method optionally comprises providing a cardiac image generated using a non-CT medical imaging technique to the DL-CNN with the 3D position and/or 3D orientation of the isotropic centroid of the aortic root and processing the cardiac image using the DL-CNN to quantify the amount of calcium in the aortic root.

In some further embodiments, the method may comprise training the DL-CNN to accurately quantify the amount of calcium in the aortic root. The training may include manually annotating a plurality of CT images and determining the amount of calcium in the aortic root to generate a training module; and training the DL-CNN using the training module for a plurality of epochs. The training may further comprise altering the orientation of the plurality of CT images and providing the altered images to the DL-CNN to determine the amount of calcium in the aortic root. The amount of calcium in the aortic root determined by the DL-CNN is then compared with the amount of calcium in the aortic root determined manually.

In some further embodiments, the training steps may further comprise providing structural parameters to the DL-CNN including a CT image pixel data, a binary mask of calcium based on thresholding, a 3D radial distance relative to the aortic valve or aortic root axis, and a projected out-of-plane 3D distance along the aortic valve or aortic root axis relative to the aortic root or valve coaptation point. Accuracy of the trained algorithm can be validated using a nine-fold cross validation technique by determining a dice coefficient, a mean average error and a mean relative error.

In some embodiments, the computer-implemented method of measuring/quantifying calcification of an aortic root using an algorithm is provided. The method involves identifying a 3D position and/or 3D orientation of a plurality of anatomical features of the aortic root on a non-contrast cardiac computed tomography image (CT image) and predicting a 3D position and/or 3D orientation of the isotropic centroid of the aortic root on the CT image. The pre-determined region surrounding the isotropic centroid is segmented into a plurality of voxels and each segmented voxel is surveyed to ascertain whether it contains calcium. These steps allow the DL-CNN to calculate an Aortic Valve Calcification (AVC) score by quantifying the number of calcium containing segmented voxels that form part of the aortic root.

In some embodiments, the algorithm comprises a multi-level convolutional neural network (ML-CNN) and a deep learning convolutional neural network (DL-CNN) and the algorithm is capable of locating an isotropic centroid of any volumetric structure on a given image. For instance, the algorithm is capable of locating an isotropic centroid of any organ or part thereof on a medical image. The isotropic centroid of the organ or part thereof could be very useful e.g. for surgical planning. In some embodiments, the isotropic centroid of the organ or part thereof could be used for identifying calcification in the organ or part thereof.

In some embodiments, a non-transitory computer readable medium having stored thereon software instructions or an algorithm that, when executed by a processor, cause the processor to perform the method of locating an isotropic centroid of an aortic root is provided. The method involves providing a non-contrast cardiac computed tomography image (CT image) to a multi-level convolutional neural network (ML-CNN). The CT image is processed by the ML-CNN to identify a 3D position and/or 3D orientation of a plurality of anatomical features of the aortic root. The 3D position and/or 3D orientation of the anatomical features allows prediction of a 3D position and/or 3D orientation of the isotropic centroid of the aortic root on the CT image.

The ML-CNN may comprise at least three convolutional blocks, at least two convolutional layers and a pooling layer. In some embodiments, the processor is trained to perform any of the computer-implemented methods recited hereinbefore, for instance, the method of localizing an isotropic centroid of an aortic root, or any organ.

In some embodiments, a non-transitory computer readable medium having stored thereon software instructions or an algorithm that, when executed by a processor, cause the processor to perform the method of quantifying an amount of calcium in an aortic root. The method involves providing a non-contrast cardiac computed tomography image (CT image) to a deep-learning convolutional neural Network (DL-CNN), wherein 3D position and/or 3D orientation of an isotropic centroid of the aortic root on the CT image is known/has been identified previously. The CT image is then processed to quantify the amount of calcium using the DL-CNN, where the DL-CNN segments a pre-determined region surrounding the isotropic centroid into a plurality of voxels; surveys each segmented voxel to ascertain whether it contains calcium; and calculates an Aortic Valve Calcification (AVC) score by quantifying the number of calcium containing segmented voxels that form part of the aortic root. In some embodiments, the DL-CNN may comprise of at least three convolutional blocks, at least two convolutional layers, at least one connecting layer and a pooling layer. In some further embodiments, the processor could be trained to perform any of the computer-implemented methods recited hereinbefore, for instance, the method of segmenting an area surrounding the isotropic centroid of any organ and quantifying calcification therein.

In some embodiments, a non-transitory computer readable medium having stored thereon software instructions/an algorithm that, when executed by a processor, cause the processor to perform the method of measuring/quantifying calcification of an aortic root using the algorithm is provided. The method involves identifying a 3D position and/or 3D orientation of a plurality of anatomical features of the aortic root on a non-contrast cardiac computed tomography image (CT image) and predicting a 3D position and/or 3D orientation of the isotropic centroid of the aortic root on the CT image. The pre-determined region surrounding the isotropic centroid is segmented into a plurality of voxels and each segmented voxel is surveyed to ascertain whether it contains calcium. These steps allow the DL-CNN to calculate an Aortic Valve Calcification (AVC) score by quantifying the number of calcium containing segmented voxels that form part of the aortic root.

In some embodiments, the algorithm may comprise a multi-level convolutional neural network (ML-CNN) and a deep learning convolutional neural network (DL-CNN), where the ML-CNN comprises at least three convolutional blocks, at least two convolutional layers, and a pooling layer and the DL-CNN comprises at least three convolutional blocks, at least two convolutional layers, at least one connecting layer and a pooling layer. In some further embodiments, the processor can be trained to perform any of the computer-implemented methods recited hereinbefore.

In some embodiments, an imaging device is provided that comprises any of the non-transitory computer-readable mediums recited hereinbefore. In some further embodiments, a system is provided that comprises any of the non-transitory computer-readable mediums recited hereinbefore. In some further embodiments, an artificially intelligent device is provided that comprises any of the non-transitory computer-readable mediums recited hereinbefore.

### BRIEF DESCRIPTION OF DRAWINGS

FIG. 1 shows an architecture of the proposed two-stage deep learning convolutional neural network based algorithm.

FIG. 2 shows sample CT images with manually annotated and multi level convolutional neural network (ML-CNN) predicted aortic valve landmarks. Manually annotated anatomical features (leaflet coaptation point [circles/crosses] and aortic root longitudinal axis [lines]) are presented in blue (B) and model predictions in red (R). The top, middle, and bottom rows show illustrative scans of patients who were in the 5^{th}, 50^{th}, and 95^{th} percentiles in model accuracy.

FIG. 3 shows sample CT images with overlay of segmented aortic calcium mask using three algorithms. Concordant voxels between the labelled segmentation and the model-predicted segmentation are blue, and discordant voxels are shown in red.

FIG. 4 provides Bland-Altman plots (A-C) and correlation plots (D-F) comparing the labeled to the model-predicted AVC scores for corresponding patients.

FIG. 5 provides receiver-operator characteristic curves for the diagnosis of severe aortic valve calcification using the three models (combined stages 1 and 2).

FIG. 6 shows a comparison of total aortic valve calcification (AVC) measured using the deep learning convolutional neural network (DL-CNN) algorithm and manually segmented AVC (A and C) and comparison of manually segmented AVC measured by different observers (B and D). Furthermore, Bland-Altman plots (A and B) and linear correlation analysis (C and D) using the coefficient of determination (R²) are presented as well.

FIG. 7 shows sample CT images with manually annotated and deep learning convolutional neural network (DL-CNN) algorithm predicted aortic valve calcium masks overlay. The top, middle, and bottom rows show scans in the 5^{th}, 50^{th}, and 95^{th} percentiles of model accuracy. The left, middle, and right columns respectively show slices from the left ventricular outflow tract (LVOT), the aortic valve annulus, and the aortic root. Segments of the images highlighted in blue, yellow, and red correspond respectively to AVC as determined by both the model and human annotators, only the human annotators, and only the algorithm. All slices are shown en face. The figure also shows the Proof of Concept Test AUCs as conducted using an initial 1,437 patient cohort.

FIG. 8 shows receiver-operator characteristics curves for severity classification stratified by patient sex.

FIG. 9 shows Bland-Altman plots (A to D) and correlation analysis (E to H) of the proposed ML-CNN and DL-CNN algorithm for automated AVC scoring in the four anatomical regions of the aortic root: the right coronary cusp (A and E), the left coronary cusp (B and F), the non-coronary cusp (C and G), and the left ventricular outflow tract (D and H).

### DETAILED DESCRIPTION

The following description is of preferred embodiments by way of example only and without limitation to the combination of features necessary for carrying the invention into effect.

All terms are intended to be understood as they would be understood by a person skilled in the art. Unless defined otherwise, all technical and scientific terms used herein have the same meaning as commonly understood by one of ordinary skill in the art to which the disclosure pertains. The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Unless otherwise defined, all technical terms used herein have the same meaning as commonly understood by one of ordinary skill in the art to which this invention belongs. As used in this specification and the appended claims, the singular forms “a”, “an”, and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

The following definitions supplement those in the art and are directed to the current application. Accordingly, the terminology used herein is for the purpose of describing particular embodiments only, and is not intended to be limiting.

### DEFINITIONS

The initialism “AI” as used in the descriptive embodiments mean “Artificial Intelligence”.

The initialism “AS” as used in the descriptive embodiments mean “Aortic Stenosis”.

The initialism “AV” as used in the descriptive embodiments mean “aortic valve”.

The initialism “AVC” as used in the descriptive embodiments mean “aortic valve calcification”.

The initialism “CNN” as used in the descriptive embodiments mean “convolutional neural network”.

The initialism “CT” as used in the descriptive embodiments mean “computed tomography”.

The initialism “DL” as used in the descriptive embodiments mean “deep-learning” i.e. artificial intelligence based deep learning algorithms and models.

The initialism “DL-CNN” as used in the descriptive embodiments mean “Deep Learning Convolutional Neural Network”.

The initialism “ECG” as used in the descriptive embodiments mean “Electrocardiogram”.

The initialism “ML-CNN” as used in the descriptive embodiments mean “Multi Level Convolutional Neural Network”.

The initialism “ROC” curve as used in the descriptive embodiments mean “Receiver Operating Characteristic” curve and the initialism “AUC” as used in the descriptive embodiments mean “Area under the curve”, more specifically, are under the ROC curve.

As used herein, the terms “artificial intelligence,” “artificial intelligence procedure”, and “artificial intelligence operation” generally refer to any system or computational procedure that takes one or more actions that may enhance or maximize a chance of successfully achieving a goal. The term “artificial intelligence” may include “machine learning” (ML) and/or “reinforcement learning” (RL).

As used herein, the terms “machine learning,” “machine learning procedure,” and “machine learning operation” generally refer to any system or analytical and/or statistical procedure that progressively improves computer performance of a task. Machine learning may include a machine learning algorithm. The machine learning algorithm may be a trained algorithm. Machine learning may comprise one or more supervised, semi-supervised, or unsupervised machine learning techniques. For example, an algorithm may be a trained algorithm that is trained through supervised learning (e.g., and various parameters are determined as weights or scaling factors). Machine learning may comprise one or more of regression analysis, regularization, classification, dimensionality reduction, ensemble learning, meta learning, association rule learning, cluster analysis, anomaly detection, deep learning, or ultra-deep learning. Machine learning may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principle component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naive Bayes, Gaussian naive Bayes, multinomial naive Bayes, hidden Markov models, hierarchical hidden Markov models, support vector machines, encoders, decoders, auto-encoders, stacked auto encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

As used herein, the term “Neural Network” refers to a system of interconnected artificial neurons (e.g., ai, a2, a3) that exchange messages between each other. The illustrated neural network has three inputs, two neurons in the hidden layer and two neurons in the output layer. The hidden layer has an activation function f(·) and the output layer has an activation function g(·). The connections have numeric weights (e.g., wn, w2i, Wi2, w3i, w22, w32, vn, v22) that are tuned during the training process, so that a properly trained network responds correctly when fed an image to recognize. The input layer processes the raw input the hidden layer processes the output from the input layer based on the weights of the connections between the input layer and the hidden layer. The output layer takes the output from the hidden layer and processes it based on the weights of the connections between the hidden layer and the output layer. The network includes multiple layers of feature-detecting neurons. Each layer has many neurons that respond to different combinations of inputs from the previous layers. These layers are constructed so that the first layer detects a set of primitive patterns in the input image data, the second layer detects patterns of patterns and the third layer detects patterns of those patterns.

A neural network model is trained using training samples before using it used to predict outputs for production samples. The quality of predictions of the trained model is assessed by using a test set of training samples that is not given as input during training. If the model correctly predicts the outputs for the test samples then it can be used in inference with high confidence. However, if the model does not correctly predict the output for test samples then the model is said to be overfitted on the training data and probably not generalized on the unseen test data.

As used herein, the term “Convolutional Neural Network” or “CNN” refers to a special type of neural network. The fundamental difference between a densely connected layer and a convolution layer is that a dense layer learns global patterns in their input feature space, whereas a convolution layer learns local patters: in the case of images, patterns found in small 2D windows of the inputs. This key characteristic gives convolutional neural networks two interesting properties: (1) the patterns they learn are translation invariant and (2) they can learn spatial hierarchies of patterns.

Regarding the first point, after learning a certain pattern in the lower-right corner of a picture, a convolution layer can recognize it anywhere, for example, in the upper-left corner. A densely connected network would have to learn the pattern anew if it appeared at a new location. This makes convolutional neural networks data efficient because they need fewer training samples to learn representations that have generalization power.

Regarding the second point, a first convolution layer can learn small local patterns such as edges, a second convolution layer will learn larger patterns made of the features of the first layers, and so on. This allows convolutional neural networks to efficiently learn increasingly complex and abstract visual concepts.

A convolutional neural network learns highly non-linear mappings by interconnecting layers of artificial neurons arranged in many different layers with activation functions that make the layers dependent. It includes one or more convolutional layers, interspersed with one or more sub-sampling layers and non-linear layers, which are typically followed by one or more fully connected layers. Each element of the convolutional neural network receives inputs from a set of features in the previous layer. The convolutional neural network learns concurrently because the neurons in the same feature map have identical weights. These local shared weights reduce the complexity of the network such that when multi-dimensional input data enters the network, the convolutional neural network avoids the complexity of data reconstruction in feature extraction and regression or classification process.

As used herein, the term “Deep neural network” refer to a type of artificial neural network that use multiple nonlinear and complex transforming layers to successively model high-level features. Deep neural networks provide feedback via backpropagation which carries the difference between observed and predicted output to adjust parameters. Deep neural networks have evolved with the availability of large training datasets, the power of parallel and distributed computing, and sophisticated training algorithms. Deep neural networks have facilitated major advances in numerous domains such as computer vision, speech recognition, and natural language processing.

Convolutional neural networks (CNNs) are a component of deep neural networks. Convolutional neural networks have succeeded particularly in image recognition with an architecture that comprises convolution layers, nonlinear layers, and pooling layers. In addition, many other emergent deep neural networks have been proposed for limited contexts, such as deep spatio-temporal neural networks, multi-dimensional recurrent neural networks, and convolutional auto.

The goal of training deep neural networks is optimization of the weight parameters in each layer, which gradually combines simpler features into complex features so that the most suitable hierarchical representations can be learned from data. A single cycle of the optimization process is organized as follows. First, given a training dataset, the forward pass sequentially computes the output in each layer and propagates the function signals forward through the network. In the final output layer, an objective loss function measures error between the inferenced outputs and the given labels. To minimize the training error, the backward pass uses the chain rale to back propagate error signals and compute gradients with respect to all weights throughout the neural network. Finally, the weight parameters are updated using optimization algorithms based on stochastic gradient descent. Whereas batch gradient descent performs parameter updates for each complete dataset, stochastic gradient descent provides stochastic approximations by performing the updates for each small set of data examples. Several optimization algorithms stem from stochastic gradient descent.

### INTRODUCTION

Aortic valve calcification (AVC) is the main pathological process leading to degenerative aortic stenosis (AS). The quantification of AVC is now recommended by clinical guidelines in patients with difficult AS evaluation and/or discordant grading which represent up to 1/33 of the AS population. The last decade has seen the development of convolutional neural networks (CNN), which have found many applications in medicine. The algorithm presented here seeks to automatically quantify AVC and localize it for each aortic valve leaflet.

The last decade has seen rapid development of deep learning (DL) methods and artificial intelligence applied to image segmentation. It was hypothesized that AVC measurement could be standardized and fully automated to improve accuracy and efficiently quantify AVC. A sequence of convolutional neural networks was developed that could automatically measure the AVC score from ECG-gated non-contrast cardiac images and identify patients with severe AS. In particular, the fully automated techniques used an ECG-gated non-contrast cardiac enhanced CT images for this purpose. A two-stage algorithm for automatic AVC quantification and segmentation is presented, and its training and evaluation in a single-center dataset of cardiac CT images with a spectrum of AVC severity are described. Implementation of an automated landmarking algorithm for the aortic valve and a hybrid 3D U-Net and ResNet architecture with multichannel geometric a priori for segmentation (MCU-ResNet) is described.

Literature evaluating the feasibility and accuracy of AVC measurement using AI is scarce. Semi-automated AVC calcium segmentation using CNNs has been evaluated in a recent study by Chang et al., This study used a modified 3D U-Net model trained in 442 CT scans and evaluated in 137 scans for the classification of severe and non-severe AVC and did not use cross-validation. The model focused on manual selection of the region surrounding the aortic valve. The study used the Rosenhek visual echocardiography based scoring system, rather than guideline directed AVC score thresholds, which is subjective and not quantitative, and included a large proportion of CT scans (242 patients) with no AVC. Chang et al. achieved an accuracy of 93% for the classification of severe and non-severe AVC.

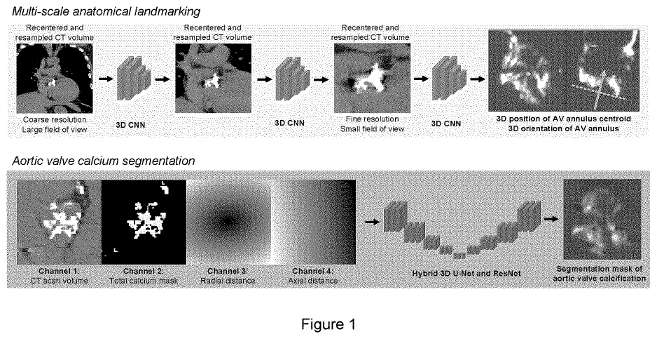

The proposed AI algorithm is formed from a combination of several CNNs generally arranged into two components or stages aiming at solving complementary tasks: (1) multi-scale anatomical feature localization (L) also referred to as ML-CNN, and (2) aortic valve calcium segmentation(S), also referred to as DL-CNN. The overall algorithm architecture is summarized graphically in FIG. 1. The first component of the algorithm takes as input the CT dataset (μ∈ [symbol] ^{MNO}) and outputs the 3D position (v∈ [symbol] ³) and orientation (\hat{n}∈ [symbol] ³:∥\hat{n}∥=1) of the aortic valve:

[v\hat{n}]=L(μ)

The second component of the algorithm(S) produces the segmentation of the aortic valve calcium mask (m∈{0,1}^{MNO}) and uses the result of the output of the first component of the algorithm to focus the segmentation on the region of the aortic valve rather than the entire CT volume, thus simplifying the task:

m=S(μ,v,ñ)

This information is finally used to calculate the AVC score using the Agatston method. The following sections focus on implementation details of each component of the algorithm.

In total, 710 patients who were referred for consideration of aortic valve intervention and who underwent an ECG-gated non-contrast enhanced CT calcium scan were included in the cohort. An additional 99 patients who underwent a cardiac CT calcium score for another indication were also included in order to provide a control cohort of patients without AVC.

The task of locating anatomical features in the aortic root was accomplished through an iterative series of three regression CNNs. At each step, the CNN took as input a CT image volume sampled with a finer spatial resolution and a more restricted field of view, while maintaining the image matrix size constant. At the first resolution step, the raw CT image data was centered about its 3D midpoint and resampled to an isotropic voxel spacing of 4 millimeters and an image matrix of 64×64×64 voxels. At this spacing, the 256 mm field of view contained nearly the entire original image, and the aortic valve center was certain to be captured. This image matrix was passed to the top-level CNN, which predicted the 3D location of the aortic valve annulus centroid as output. At the final step, the CNNs predicted the 3D location of the leaflet coaptation central point, and the 3D orientation of the aortic valve annulus viewed en face expressed as a 3D normal vector

Between the first and second CNNs, the raw image was centered about the predicted valve annulus centroid and resized to an isotropic spacing of 2 mm per voxel. This spacing was smaller than the previous spacing; hence, any errors in predicting the valve center by the top-level CNN were magnified in this new resized image. The second CNN took as input the resized image volume and again predicted the 3D location of the aortic valve annulus centroid as output. While this process of recursively locating the valve center and using its location to recenter and resize to a smaller field of view could be performed indefinitely, there could be practical limitations to how many iterations were desired. The revised 3D location of the aortic valve annulus centroid was once again used to recenter and resize the raw image down to a resolution of 1 millimeter per voxel. This resized image was passed to two final CNNs, which predicted (1) the 3D locations of anatomical features of interest, and (2) the 3D orientation of the aortic valve annulus viewed en face.

Each CNN consisted of identical architectures, with the exception for the output layer, given the varying number of points predicted. The CNN architectures contain three convolutional blocks, composed of two convolutional layers followed by a max pooling layer. The convolutions were configured to be 3D. After the convolutional blocks, the data was flattened and passed through a fully connected layer before finally reaching the output layer. In total, each model contains 34,426,037 trainable parameters, which corresponds to 395 MB of disk space. The models were trained for 13.5 hours, which is equivalent to training over 200,000 scans and data augmentation included random variation in 3D translation, 3D rotation, and scale.

The task of determining whether a voxel containing calcium (with an CT attenuation value ≥130 HU) is part of the aortic valve or not was solved using a segmentation algorithm. A novel machine learning architecture, combining the block-level design of the ResNet model with the overall structure of a U-net was used. ResNets are deep learning models, capable of learning complex image structures, and U-nets are commonly used for image segmentation tasks; a blend of the two is ideal for this task.

The model involved recentering the raw CT image about isotropic centroid position to ensure all calcium would be within the field of view. The raw CT images were of shape n×512×512, where n is the number of CT slices; in addition to recentering, the field of view was reduced to 32×128×128, to capture the full valve while minimizing the amount of non-valvular calcium. In contrast to the previous task, the anisotropic resolution of the image volume was retained. This recentered and resized image was used as input to the segmentation algorithm, which returned a mask of aortic valve calcium. This output mask was an array of the same shape as the input image, where the value of each voxel was 1 if the voxel was predicted to be AVC, and 0 otherwise.

In total, the segmentation algorithm contained 15,920,769 trainable parameters, which corresponded to 183 MB of disk space. The model was trained for 4.5 hours, which is equivalent to training over 20,000 scans.

The aortic valve calcium score was computed as described above including only the segmented voxels. Using the output of the aortic root feature localization algorithm, the segmented voxels between the three aortic valve cusps and the LVOT were also separated in order to calculate a calcium score for each of these regions.

Automating the quantification process of AVC calcification may result time savings users during image analysis by radiologists or cardiologists. There may also be improvements in aortic valve score accuracy for less experienced users.

### EMBODIMENTS

Without wishing to be bound by theory or experimental results, the following paragraphs describe the nature of the invention by way of examples only. The experiments, methods, specific examples or embodiments described should not be construed as limiting the scope of the invention. A person skilled in the art would readily understand and appreciate that other experiments, examples, applications, methods not specifically described also form part of the invention.

In an embodiment of the invention, a computer-implemented method of locating an isotropic centroid of an aortic root is provided. The method involves providing a non-contrast cardiac computed tomography image (CT image) to a multi-level convolutional neural network (ML-CNN). The CT image is processed by the ML-CNN to identify a 3D position and/or 3D orientation of a plurality of anatomical features of the aortic root. The 3D position and/or 3D orientation of the anatomical features allows prediction of a 3D position and/or 3D orientation of the isotropic centroid of the aortic root on the CT image. In a further embodiment, the ML-CNN identifies the 3D position and a 3D orientation of the plurality of anatomical features of the aortic root to predict the 3D position and a 3D orientation of the isotropic centroid of the aortic root on the CT image.

In a further embodiment, the ML-CNN predicts the 3D position and/or 3D orientation of the isotropic centroid by processing/surveying the CT image to first identify the image 3D midpoint. This is followed by refining the spatial resolution and restricting the field of view to focus on the area surrounding the 3D midpoint of the CT image with an isotropic voxel spacing that captures the entire aortic root. It is pertinent to note that the image matrix is kept constant and remain unchanged unlike the resolution and field of view. Once the ML-CNN is focused on the area surrounding the 3D midpoint, the 3D position and/or 3D orientation of the plurality of anatomical features of the aortic root are identified which allows prediction of the 3D position and/or 3D orientation of the isotropic centroid of the aortic root.

In some embodiments, the steps of identifying the 3D position and/or 3D orientation of the plurality of anatomical features of the aortic root and its isotropic centroid are repeated multiple times by the ML-CNN by further refining the spatial resolution and restricting the field of view each time while keeping the image matrix constant. After every prediction, the ML-CNN uses a previous prediction of the 3D position and/or 3D orientation of the isotropic centroid of the aortic root as a starting point before predicting a new 3D position and/or 3D orientation of the isotropic centroid.

The plurality of anatomical features that could be identified by the ML-CNN can be an aortic valve annulus centroid, at least one aortic valve commissure, an aortic leaflet coaptation point, and at least one coronary ostia.

In some embodiments, the plurality of anatomical features are identified by determining the axial distance between each anatomical feature. In some further embodiments, the plurality of anatomical features are identified as geometric points on the CT image by using a different colour for each anatomical feature.

In some embodiments, the ML-CNN restricts the field of view to an isotropic voxel spacing of 4 mm, or 2 mm, or 1 mm. In some embodiments, the field of view could be restricted to any isotropic voxel spacing as long as the image matrix is kept constant. The ML-CNN can maintain the CT image size matrix to 64 cube voxel, 32 cube voxel, 24 cube voxel, 16 cube voxel, 8 cube voxel or 1 cube voxel.

In some embodiments, the ML-CNN may predict the isotropic centroid of each aortic leaflet separately. In some embodiments, the ML-CNN may predicts an edge of the left cusp, right cusp, non-coronary cusp and left ventricular outflow tract [LVOT] regions on the CT image.

In some further embodiments, the computer-implemented method and the ML-CNN algorithm may involve use a cardiac image generated using a non-CT medical imaging technique. In such embodiments, ML-CNN processes the cardiac image to predict the 3D position and/or 3D orientation of the isotropic centroid of the aortic root.

In some embodiments, the ML-CNN may be trained to accurately identify 3D position and/or 3D orientation of the plurality of anatomical features of the aortic root and to accurately predict the 3D position and/or 3D orientation of the isotropic centroid of the aortic root based on the 3D position and/or 3D orientation of the anatomical features. The training may involve manually annotating a plurality of CT images and determining the 3D position and/or 3D orientation of the isotropic centroid of the aortic root to generate a training module. Once the manual annotations are complete, the ML-CNN can be trained using the training module for a plurality of epochs. In some embodiments, the training may require altering the orientation of the plurality of CT images and allowing the ML-CNN to determine the isotropic centroid of the aortic root after the CT images are altered. Once the 3D position and/or 3D orientation of the isotropic centroid of the aortic root is predicted by the ML-CNN, it can be compared with the 3D position and/or 3D orientation that was predicted during manual annotations. The alteration of CT images may include random alteration of the orientation of the plurality of images by varying the image field of view, by rotating the image or by scaling-down pixel spacing of the image. The training may also include testing the ML-CNN by providing a new CT image to the ML-CNN for predicting the 3D position and/or 3D orientation of the isotropic centroid of the aortic root, wherein the isotropic centroid of the aortic root on the CT image is already known/has been identified previously by manual annotation. Thereafter, accuracy of the ML-CNN's prediction can be determined by comparing the 3D position and/or 3D orientation of the isotropic centroid predicted by the ML-CNN with the 3D position and/or 3D orientation identified previously. In some embodiments, the accuracy of the ML-CNN is validated using a nine-fold cross validation technique by determining a 3D position and/or 3D orientation error and a 3D angular error.

In some embodiments, the method is used to locate 3D position and/or 3D orientation of an isotropic centroid of any organ or part thereof on a medical image and the 3D position and/or 3D orientation of the isotropic centroid of the organ or part thereof could be useful for surgical planning.

In certain embodiment, the method may optionally comprise providing a plurality of non-contrast cardiac computed tomography images (CT images) defined by (μ∈ [symbol] ^{MNO}) to the ML-CNN (defined as L). The ML-CNN then processes the plurality of CT images by identifying the 3D position and/or the 3D orientation of the plurality of anatomical features of the aortic root to predict the 3D position defined by (v∈ [symbol] ³) and the 3D orientation defined by (\hat{n}∈ [symbol] ³: ∥\hat{n}∥=1), where the 3D position and/or the 3D orientation provide an anatomical landmark defined by ([v \hat{n}]=L(μ)) and a spatial position defined by xᵢⱼₖ of the isotropic centroid of the aortic root on the CT images. In the above equation, v is an annulus centroid position and \hat{n} is a unit normal vector of an aortic annulus plane of the anatomical landmark, and the CT images have an image matrix of dimension M×N×O, with positional indices i, j, k in x, y, z directions respectively.

In some embodiments, a computer-implemented method of quantifying an amount of calcium in an aortic root is provided. The method involves providing a non-contrast cardiac computed tomography image (CT image) to a deep-learning convolutional neural Network (DL-CNN), wherein 3D position and/or 3D orientation of an isotropic centroid of the aortic root on the CT image is known/has been identified previously. The CT image is then processed to quantify the amount of calcium using the DL-CNN, where the DL-CNN segments a pre-determined region surrounding the isotropic centroid into a plurality of voxels; surveys each segmented voxel to ascertain whether it contains calcium; and calculates an Aortic Valve Calcification (AVC) score by quantifying the number of calcium containing segmented voxels that form part of the aortic root.

In some embodiments, the CT image provided to the DL-CNN has a field of view defined by n×512×512, where n is the number of CT slices. The DL-CNN restricts the field of view to 32×128×128, 16×64×64, 8×32×32, 4×16×16 or 2×4×4 prior to segmenting the pre-determined region to capture the entire aortic root on the CT image.

In some embodiments, the DL-CNN assigns a score of 1 to a segmented voxel that contains calcium and a score of 0 to a segmented voxel that does not contain calcium. It is pertinent to note that DL-CNN does not take into account a segmented voxel that contains calcium but does not form part of the aortic valve. DL-CNN identifies the segmented voxel containing calcium by determining a CT attenuation value and a CT attenuation value of ≥130 HU indicates that the segmented voxel contains calcium.

In some embodiments, the DL-CNN creates a mask that displays the segmented voxels that were identifies as containing calcium. In some further embodiments, the DL-CNN quantifies the amount of calcium of each aortic leaflet separately. In some further embodiments, the DL-CNN quantifies the amount of calcium of left cusp, right cusp, non-coronary cusp and left ventricular outflow tract [LVOT] separately.

The AVC score assigned by DL-CNN is an indicator or various medical conditions. For instance, a higher AVC score is an indicator of cardiac conditions, or an indicator of Aortic Stenosis. In some embodiments, the proposed segmentation and quantification method can be carried out to track the progress of Aortic Stenosis. A higher AVC score could also be an indicator of periprocedural complications in patients undergoing transcatheter aortic valve implantation (TAVI). The presence of calcium/a higher amount of calcium in the LVOT is an indicator of need for pacemaker implantation or paravalvular leak after TAVI. In some embodiment, the method may be employed to quantify the amount of calcium in any organ or a part thereof on a medical image.

In some embodiments, the DL-CNN may be composed of more than one neural network models. For instance, the DL-CNN could be a combination of two or more neural network models e.g. the DL-CNN could be a combination of a U-Net model and a ResNet model.

In some further embodiments, the method optionally comprises providing a cardiac image generated using a non-CT medical imaging technique to the DL-CNN with the 3D position and/or 3D orientation of the isotropic centroid of the aortic root and processing the cardiac image using the DL-CNN to quantify the amount of calcium in the aortic root.

In some further embodiments, the method may comprise training the DL-CNN to accurately quantify the amount of calcium in the aortic root. The training may include manually annotating a plurality of CT images and determining the amount of calcium in the aortic root to generate a training module; and training the DL-CNN using the training module for a plurality of epochs. The training may further comprise altering the orientation of the plurality of CT images and providing the altered images to the DL-CNN to determine the amount of calcium in the aortic root. The amount of calcium in the aortic root determined by the DL-CNN is then compared with the amount of calcium in the aortic root determined manually.

In some further embodiments, the training steps may further comprise providing structural parameters to the DL-CNN including a CT image pixel data, a binary mask of calcium based on thresholding, a 3D radial distance relative to the aortic valve or aortic root axis, and a projected out-of-plane 3D distance along the aortic valve or aortic root axis relative to the aortic valve or aortic root coaptation point. Accuracy of the trained algorithm can be validated using a nine-fold cross validation technique by determining a dice coefficient, a mean average error and a mean relative error.

The mean average error may be determined using a formula

$${σ}_{MAE} = \frac{1}{N} {\sum }_{n = 0}^{N - 1} \vert {c}_{n} - {\tilde{c}}_{n} \vert$$

and the mean relative error may be determined using a formula

$${σ}_{MRE} = \frac{2}{N} {\sum }_{n = 0}^{N - 1} \frac{\vert {c}_{n} - {\tilde{c}}_{n} \vert}{\vert {c}_{n} + {\tilde{c}}_{n} \vert}$$

where the sum is over N patients, cₙ is a predicted AVC score, and \tilde{c}ₙ is a labeled AVC score.

The various structural parameters may be defined as noted below. The CT pixel data may be defined by μ∈ [symbol] ^{MNO} with an image matrix of dimension M×N×O, and positional indices i, j, k in the x, y, z directions respectively. The binary mask may be defined by t∈{0,1}^{MNO} of all calcium based on thresholding where:

$${t}_{i j k} = \{ \begin{matrix}0 & {μ}_{i j k} < 130 HU \\ 1 & {μ}_{i j k} \geq 130 HU\end{matrix} .$$

The 3D radial distance relative to the aortic root or aortic valve axis may be defined by r∈ [symbol] ^{MNO} where:

rᵢⱼₖ(v,\hat{n})=∥(xᵢⱼₖ−v)−\hat{n}((xᵢⱼₖ−v)·\hat{n})∥.

The projected out-of-plane 3D distance which is relative to the aortic valve or aortic root annular plane may be defined by d∈ [symbol] ^{MNO} where:

dᵢⱼₖ(v,\hat{n})=(x_{i,j,k}−v)·\hat{n};

In the above equation xᵢⱼₖ is a spatial position at the center of voxel i, j, k, v is the annulus centroid position, \hat{n} is a unit normal vector of the aortic annulus plane.

Using the above structural parameter values, the DL-CNN (defined as S) provides a segmentation of the aortic root calcium mask (m) defined by

$$m = S ( μ , v , \hat{n} ) = {S}_{n n} ( [ \begin{matrix}μ \\ t ( μ ) \\ r ( v , \hat{n} ) \\ d ( v , \hat{n} )\end{matrix} ] )$$

It is pertinent to note that m∈{0,1}^{MNO} is an array of the same shape as the CT images, where the DL-CNN assigns a score of 1 to a segmented voxel that contains calcium and a score of 0 to a segmented voxel that does not contain calcium.

In some embodiments, the computer-implemented method of measuring/quantifying calcification of an aortic root using an algorithm is provided. The method involves identifying a 3D position and/or 3D orientation of a plurality of anatomical features of the aortic root on a non-contrast cardiac computed tomography image (CT image) and predicting a 3D position and/or 3D orientation of the isotropic centroid of the aortic root on the CT image. The pre-determined region surrounding the isotropic centroid is segmented into a plurality of voxels and each segmented voxel is surveyed to ascertain whether it contains calcium. These steps allow the DL-CNN to calculate an Aortic Valve Calcification (AVC) score by quantifying the number of calcium containing segmented voxels that form part of the aortic root.

In some embodiments, the algorithm comprises a multi-level convolutional neural network (ML-CNN) and a deep learning convolutional neural network (DL-CNN) and the algorithm is capable of locating an isotropic centroid of any volumetric structure on a given image. For instance, the algorithm is capable of locating an isotropic centroid of any organ or part thereof on a medical image. The isotropic centroid of the organ or part thereof could be very useful e.g. for surgical planning. In some embodiments, the isotropic centroid of the organ or part thereof could be used for identifying calcification in the organ or part thereof. In some embodiments, the method may comprise a combination of the method steps defined for ML-CNN and DL-CNN above.

In some embodiments, a non-transitory computer readable medium having stored thereon software instructions or an algorithm that, when executed by a processor, cause the processor to perform the method of locating an isotropic centroid of an aortic root is provided. The method involves providing a non-contrast cardiac computed tomography image (CT image) to a multi-level convolutional neural network (ML-CNN). The CT image is processed by the ML-CNN to identify a 3D position and/or 3D orientation of a plurality of anatomical features of the aortic root. The 3D position and/or 3D orientation of the anatomical features allows prediction of a 3D position and/or 3D orientation of the isotropic centroid of the aortic root on the CT image. In some embodiments, an imaging device may comprise the above non-transitory computer-readable medium. In some further embodiments, the non-transitory computer-readable medium may be comprised in a system. In some further embodiments, an artificially intelligent device may comprise the non-transitory computer-readable medium.

The ML-CNN may comprise at least three convolutional blocks, at least two convolutional layers and a pooling layer. In some embodiments, the processor is trained to perform any of the computer-implemented methods recited hereinbefore, for instance, the method of localizing an isotropic centroid of an aortic root, or any organ.

For instance the processor may be trained to perform the steps of:
a computer-implemented method of locating an isotropic centroid of an aortic root by providing a non-contrast cardiac computed tomography image (CT image) to a multi-level convolutional neural network (ML-CNN). The CT image is processed by the ML-CNN to identify a 3D position and/or 3D orientation of a plurality of anatomical features of the aortic root. The 3D position and/or 3D orientation of the anatomical features allows prediction of a 3D position and/or 3D orientation of the isotropic centroid of the aortic root on the CT image. In a further embodiment, the ML-CNN identifies the 3D position and a 3D orientation of the plurality of anatomical features of the aortic root to predict the 3D position and a 3D orientation of the isotropic centroid of the aortic root on the CT image.
a computer-implemented method of predicting the 3D position and/or 3D orientation of the isotropic centroid by the ML-CNN by processing/surveying the CT image to first identify the image 3D midpoint. This is followed by refining the spatial resolution and restricting the field of view to focus on the area surrounding the 3D midpoint of the CT image with an isotropic voxel spacing that captures the entire aortic root. It is pertinent to note that the image matrix is kept constant and remain unchanged unlike the resolution and field of view. Once the ML-CNN is focused on the area surrounding the 3D midpoint, the 3D position and/or 3D orientation of the plurality of anatomical features of the aortic root are identified which allows prediction of the 3D position and/or 3D orientation of the isotropic centroid of the aortic root.
identifying the 3D position and/or 3D orientation of the plurality of anatomical features of the aortic root and its isotropic centroid may be repeated multiple times by the ML-CNN by further refining the spatial resolution and restricting the field of view each time while keeping the image matrix constant. After every prediction, the ML-CNN uses a previous prediction of the 3D position and/or 3D orientation of the isotropic centroid of the aortic root as a starting point before predicting a new 3D position and/or 3D orientation of the isotropic centroid. The plurality of anatomical features that could be identified by the ML-CNN can be an aortic valve annulus centroid, at least one aortic valve commissure, an aortic leaflet coaptation point, and at least one coronary ostia.
identifying the plurality of anatomical features by determining the axial distance between each anatomical feature. In some further embodiments, the plurality of anatomical features are identified as geometric points on the CT image by using a different colour for each anatomical feature.
restricting the field of view by the ML-CNN to an isotropic voxel spacing of 4 mm, or 2 mm, or 1 mm. In some embodiments, the field of view could be restricted to any isotropic voxel spacing as long as the image matrix is kept constant. The ML-CNN can maintain the CT image size matrix to 64 cube voxel, 32 cube voxel, 24 cube voxel, 16 cube voxel, 8 cube voxel or 1 cube voxel.
predicting the isotropic centroid of each aortic leaflet separately by the ML-CNN. In some embodiments, the ML-CNN may predicts an edge of the left cusp, right cusp, non-coronary cusp and left ventricular outflow tract [LVOT] regions on the CT image.

In some further embodiments, the processor may be trained to perform each of the above computer-implemented method steps and the ML-CNN algorithm may involve use a cardiac image generated using a non-CT medical imaging technique. In such embodiments, ML-CNN processes the cardiac image to predict the 3D position and/or 3D orientation of the isotropic centroid of the aortic root.

In some embodiments, the ML-CNN may be trained to accurately identify 3D position and/or 3D orientation of the plurality of anatomical features of the aortic root and to accurately predict the 3D position and/or 3D orientation of the isotropic centroid of the aortic root based on the 3D position and/or 3D orientation of the anatomical features. The training may involve manually annotating a plurality of CT images and determining the 3D position and/or 3D orientation of the isotropic centroid of the aortic root to generate a training module. Once the manual annotations are complete, the ML-CNN can be trained using the training module for a plurality of epochs. In some embodiments, the training may require altering the orientation of the plurality of CT images and allowing the ML-CNN to determine the isotropic centroid of the aortic root after the CT images are altered. Once the 3D position and/or 3D orientation of the isotropic centroid of the aortic root is predicted by the ML-CNN, it can be compared with the 3D position and/or 3D orientation that was predicted during manual annotations. The alteration of CT images may include random alteration of the orientation of the plurality of images by varying the image field of view, by rotating the image or by scaling-down pixel spacing of the image. The training may also include testing the ML-CNN by providing a new CT image to the ML-CNN for predicting the 3D position and/or 3D orientation of the isotropic centroid of the aortic root, wherein the isotropic centroid of the aortic root on the CT image is already known/has been identified previously by manual annotation. Thereafter, accuracy of the ML-CNN's prediction can be determined by comparing the 3D position and/or 3D orientation of the isotropic centroid predicted by the ML-CNN with the 3D position and/or 3D orientation identified previously. In some embodiments, the accuracy of the ML-CNN is validated using a nine-fold cross validation technique by determining a 3D position and/or 3D orientation error and a 3D angular error.

In some embodiments, the processor may be trained to perform the method to locate 3D position and/or 3D orientation of an isotropic centroid of any organ or part thereof on a medical image and the 3D position and/or 3D orientation of the isotropic centroid of the organ or part thereof could be useful for surgical planning.

In certain embodiment, the processor may be trained to perform the method steps of optionally providing a plurality of non-contrast cardiac computed tomography images (CT images) defined by (μ∈ [symbol] ^{MNO}) to the ML-CNN (defined as L). The ML-CNN then processes the plurality of CT images by identifying the 3D position and/or the 3D orientation of the plurality of anatomical features of the aortic root to predict the 3D position defined by (v∈ [symbol] ³) and the 3D orientation defined by (\hat{n}∈ [symbol] ³: ∥\hat{n}∥=1), where the 3D position and/or the 3D orientation provide an anatomical landmark defined by ([v \hat{n}]=L(μ)) and a spatial position defined by xᵢⱼₖ of the isotropic centroid of the aortic root on the CT images. In the above equation, v is an annulus centroid position and \hat{n} is a unit normal vector of an aortic annulus plane of the anatomical landmark, and the CT images have an image matrix of dimension M×N×O, with positional indices i, j, k in x, y, z directions respectively.

In some embodiments, a non-transitory computer readable medium having stored thereon software instructions or an algorithm that, when executed by a processor, cause the processor to perform the method of quantifying an amount of calcium in an aortic root. The method involves providing a non-contrast cardiac computed tomography image (CT image) to a deep-learning convolutional neural Network (DL-CNN), wherein 3D position and/or 3D orientation of an isotropic centroid of the aortic root on the CT image is known/has been identified previously. The CT image is then processed to quantify the amount of calcium using the DL-CNN, where the DL-CNN segments a pre-determined region surrounding the isotropic centroid into a plurality of voxels; surveys each segmented voxel to ascertain whether it contains calcium; and calculates an Aortic Valve Calcification (AVC) score by quantifying the number of calcium containing segmented voxels that form part of the aortic root. In some embodiments, the DL-CNN may comprise of at least three convolutional blocks, at least two convolutional layers, at least one connecting layer and a pooling layer. In some further embodiments, the processor could be trained to perform any of the computer-implemented methods recited hereinbefore, for instance, the method of segmenting an area surrounding the isotropic centroid of any organ and quantifying calcification therein. In some embodiments, an imaging device may comprise the above non-transitory computer-readable medium. In some further embodiments, the non-transitory computer-readable medium may be comprised in a system. In some further embodiments, an artificially intelligent device may comprise the non-transitory computer-readable medium.

The imaging device described in the application may consists a main magnet formed by superconducting coils, gradient coils, radiofrequency (RF) coils, and computer systems. The device may also include a scanner, a scanning unit which may include one or more X-ray tubes, Photon detectors, shielding elements, a generator, an image processor, and a console (control unit). The computer or the control unit may be equipped with the non-transitory readable medium including the ML-CNN/DL-CNN algorithms as described hereinbefore. The imaging device may also be equipped with an artificially intelligent module to convert it to an artificially intelligent device.

In some embodiments, a non-transitory computer readable medium having stored thereon software instructions/an algorithm that, when executed by a processor, cause the processor to perform the method of measuring/quantifying calcification of an aortic root using the algorithm is provided. The method involves identifying a 3D position and/or 3D orientation of a plurality of anatomical features of the aortic root on a non-contrast cardiac computed tomography image (CT image) and predicting a 3D position and/or 3D orientation of the isotropic centroid of the aortic root on the CT image. The pre-determined region surrounding the isotropic centroid is segmented into a plurality of voxels and each segmented voxel is surveyed to ascertain whether it contains calcium. These steps allow the DL-CNN to calculate an Aortic Valve Calcification (AVC) score by quantifying the number of calcium containing segmented voxels that form part of the aortic root.

The DL-CNN and the processor could be trained to perform the following method steps:
a computer-implemented method of quantifying an amount of calcium in an aortic: The method involves providing a non-contrast cardiac computed tomography image (CT image) to a deep-learning convolutional neural Network (DL-CNN), wherein 3D position and/or 3D orientation of an isotropic centroid of the aortic root on the CT image is known/has been identified previously. The CT image is then processed to quantify the amount of calcium using the DL-CNN, where the DL-CNN segments a pre-determined region surrounding the isotropic centroid into a plurality of voxels; surveys each segmented voxel to ascertain whether it contains calcium; and calculates an Aortic Valve Calcification (AVC) score by quantifying the number of calcium containing segmented voxels that form part of the aortic root.
optionally, the CT image provided to the DL-CNN has a field of view defined by n×512×512, where n is the number of CT slices. The DL-CNN restricts the field of view to 32×128×128, 16×64×64, 8×32×32, 4×16×16 or 2×4×4 prior to segmenting the pre-determined region to capture the entire aortic root on the CT image.
optionally, the DL-CNN assigns a score of 1 to a segmented voxel that contains calcium and a score of 0 to a segmented voxel that does not contain calcium. It is pertinent to note that DL-CNN does not take into account a segmented voxel that contains calcium but does not form part of the aortic valve. DL-CNN identifies the segmented voxel containing calcium by determining a CT attenuation value and a CT attenuation value of ≥130 HU indicates that the segmented voxel contains calcium.
optionally, the DL-CNN creates a mask that displays the segmented voxels that were identifies as containing calcium. In some further embodiments, the DL-CNN quantifies the amount of calcium of each aortic leaflet separately. In some further embodiments, the DL-CNN quantifies the amount of calcium of left cusp, right cusp, non-coronary cusp and left ventricular outflow tract [LVOT] separately.
optionally, the DL-CNN may be composed of more than one neural network models. For instance, the DL-CNN could be a combination of two or more neural network models e.g. the DL-CNN could be a combination of a U-Net model and a ResNet model.
optionally, the method optionally comprises providing a cardiac image generated using a non-CT medical imaging technique to the DL-CNN with the 3D position and/or 3D orientation of the isotropic centroid of the aortic root and processing the cardiac image using the DL-CNN to quantify the amount of calcium in the aortic root.
optionally, the method may comprise training the DL-CNN to accurately quantify the amount of calcium in the aortic root. The training may include manually annotating a plurality of CT images and determining the amount of calcium in the aortic root to generate a training module; and training the DL-CNN using the training module for a plurality of epochs. The training may further comprise altering the orientation of the plurality of CT images and providing the altered images to the DL-CNN to determine the amount of calcium in the aortic root. The amount of calcium in the aortic root determined by the DL-CNN is then compared with the amount of calcium in the aortic root determined manually.
optionally, the training steps may further comprise providing structural parameters to the DL-CNN including a CT image pixel data, a binary mask of calcium based on thresholding, a 3D radial distance relative to the aortic valve or aortic root axis, and a projected out-of-plane 3D distance along the aortic valve or aortic root axis relative to the aortic valve or aortic root coaptation point. Accuracy of the trained algorithm can be validated using a nine-fold cross validation technique by determining a dice coefficient, a mean average error and a mean relative error.

The mean average error may be determined using a formula

$${σ}_{MAE} = \frac{1}{N} {\sum }_{n = 0}^{N - 1} \vert {c}_{n} - {\tilde{c}}_{n} \vert$$

and the mean relative error may be determined using a formula

$${σ}_{MRE} = \frac{2}{N} {\sum }_{n = 0}^{N - 1} \frac{\vert {c}_{n} - {\tilde{c}}_{n} \vert}{\vert {c}_{n} + {\tilde{c}}_{n} \vert}$$

where the sum is over N patients, cₙ is a predicted AVC score, and \tilde{c}ₙ is a labeled AVC score.

The various structural parameters may be defined as noted below. The CT pixel data may be defined by μ∈ [symbol] ^{MNO} with an image matrix of dimension M×N×O, and positional indices i, j, k in the x, y, z directions respectively. The binary mask may be defined by t∈{0,1}^{MNO} of all calcium based on thresholding where:

$${t}_{i j k} = \{ \begin{matrix}0 & {μ}_{i j k} < 130 HU \\ 1 & {μ}_{i j k} \geq 130 HU\end{matrix} .$$

The 3D radial distance relative to the aortic root or aortic valve axis may be defined by r∈ [symbol] ^{MNO} where:

rᵢⱼₖ(v,\hat{n})=∥(xᵢⱼₖ−v)−\hat{n}(xᵢⱼₖ−v)·\hat{n})∥.

The projected out-of-plane 3D distance which is relative to the aortic valve or aortic root annular plane may be defined by d∈ [symbol] ^{MNO} where:

dᵢⱼₖ(v,\hat{n})=(x_{i,j,k}−v)·\hat{n};

In the above equation xᵢⱼₖ is a spatial position at the center of voxel i, j, k, v is the annulus centroid position, \hat{n} is a unit normal vector of the aortic annulus plane.

Using the above structural parameter values, the DL-CNN (defined as S) provides a segmentation of the aortic root calcium mask (m) defined by

$$m = S ( μ , v , \hat{n} ) = {S}_{n n} ( [ \begin{matrix}μ \\ t ( μ ) \\ r ( v , \hat{n} ) \\ d ( v , \hat{n} )\end{matrix} ] )$$

It is pertinent to note that m∈{0,1}^{MNO} is an array of the same shape as the CT images, where the DL-CNN assigns a score of 1 to a segmented voxel that contains calcium and a score of 0 to a segmented voxel that does not contain calcium.

In some embodiments, the algorithm may comprise a multi-level convolutional neural network (ML-CNN) and a deep learning convolutional neural network (DL-CNN), where the ML-CNN comprises at least three convolutional blocks, at least two convolutional layers, and a pooling layer and the DL-CNN comprises at least three convolutional blocks, at least two convolutional layers, at least one connecting layer and a pooling layer. In some further embodiments, the processor can be trained to perform any of the computer-implemented methods recited hereinbefore.

For instance the processor may be trained to perform the steps of:
measuring/quantifying calcification of an aortic root using an algorithm: The method involves identifying a 3D position and/or 3D orientation of a plurality of anatomical features of the aortic root on a non-contrast cardiac computed tomography image (CT image) and predicting a 3D position and/or 3D orientation of the isotropic centroid of the aortic root on the CT image. The pre-determined region surrounding the isotropic centroid is segmented into a plurality of voxels and each segmented voxel is surveyed to ascertain whether it contains calcium. These steps allow the DL-CNN to calculate an Aortic Valve Calcification (AVC) score by quantifying the number of calcium containing segmented voxels that form part of the aortic root.

The convolutional neural networks described herein can be trained using a variety of optimization algorithms and deep learning methods available in the art. For instance, the algorithms can be optimized by designing a training problem with a plurality of trainable parameters and could be trained using various optimization techniques, some non-limiting examples of which are Gradient descent method, Newton method, Stochastic, descent method, Mini-batch gradient method, Nesterov accelerated gradient method, Adagrad, AdaDelta, Adaptive moment estimation, Conjugate gradient method, Quasi-Newton method, and Levenberg-Marquardt algorithm.

Additionally, a person skilled in the art would appreciate that although the localization and segmentation technique is designed using convolutional neural networks, the same algorithm would work with any other neural network known in the art. Some non-limiting examples of which would be Long Short Term Memory Networks (LSTMs), Recurrent Neural Networks (RNNs), Generative Adversarial Networks (GANs), Radial Basis Function Networks (RBFNs), Multilayer Perceptrons (MLPs), Self Organizing Maps (SOMs), Deep Belief Networks (DBNs), Restricted Boltzmann Machines (RBMs) and Autoencoders.

### Cohort

A specific cohort of patients who were referred for a transcatheter or surgical aortic valve replacement at a single Canadian academic hospital and who underwent a clinically indicated CT calcium score between 2008 and 2020 were included in the study. As negative controls, the cohort was enriched with patients without significant aortic valve calcium who underwent a cardiac CT calcium score for assessment of coronary artery disease. Clinical data were extracted from the clinical electronic medical record.

Additionally, Echocardiographic reports were mined for aortic stenosis severity indices (aortic valve area, mean gradient) and aortic, mitral and tricuspid regurgitation severity. Invasive angiography reports were mined for the number of major coronary arteries with ≥50% stenosis. Symptoms severity based on the NYHA functional class was extracted from clinical notes. Patients with a prior aortic valve replacement and Sievers type 0 bicuspid aortic valve were excluded from the database. Furthermore, patients with CT images of poor quality due to truncation, beam hardening, and/or motion artifacts were also excluded. Approval was obtained for the study from the Research Ethics Board at the institution. Patient consent was waived given the retrospective nature of this analysis.

### CT Analysis and Annotation

The CT scans were first deidentified and imported in a custom analysis software allowing for manual selection of calcium and the creation of a mask defining the regions of AVC. Calcification was defined as an area of voxels ≥1 mm² with a CT attenuation value ≥130 HU, the accepted threshold for tube voltage of 120 kVp. AVC was calculated using the Agatston method and expressed in arbitrary units (AU) (7). Any calcium above the sinotubular junction, calcium affecting the right or left coronary artery ostia, and calcium further than 5 or 10 mm on the ventricular aspect of the aortic valve annular plane was excluded. These restrictions aimed respectively at excluding calcification of the ascending aorta, coronary arteries, and mitral annulus.

Each CT volume was annotated with seven 3D markers, the aortic valve annulus centroid, the three aortic valve commissures (or the two commissures and the raphe in case of bicuspid aortic valve), the leaflet coaptation center point, and two coronary ostia. The aortic valve was further divided into four regions of interest (left, right and non-coronary cusp and left ventricular outflow tract [LVOT]). The dataset was split between three cardiologists with training in CT analysis, who each performed the segmentation for one third of the images; a fourth cardiologist independently analyzed the entire dataset to evaluate reproducibility of calcium quantification between human annotators.

### Proposed Convolutional Neural Networks

The proposed two stage algorithm, for instance ML-CNN and DL-CNN (e.g. MCU-ResNet) is composed of several convolutional neural networks (CNNs), and is generally arranged into two components or stages aimed at solving complementary tasks: (1) anatomical feature landmarking and (2) aortic valve calcium segmentation.

The overall algorithm architecture is summarized graphically in FIG. 1. FIG. 1 shows an architecture of the proposed two-stage deep learning convolutional neural network based algorithm. The first component of the algorithm takes as input the CT dataset and outputs the 3D position and/or 3D orientation and orientation of the aortic valve and root. The second component of the algorithm involves the segmentation of the aortic valve and uses the result of the first component of the algorithm to focus the segmentation on the region of the aortic valve rather than the entire CT volume, thus simplifying the task. This information is finally used to calculate the AVC score. The following sections focus on implementation details of each component of the algorithm.

### Aortic Root Feature Landmarking

This component of the algorithm is based on a method previously studied in contrast-enhanced CT (8). In one embodiment, the task of locating anatomical features in the aortic root was accomplished through an iterative series of three regression CNNs. A person skilled in the art would readily understand that any number of regression, multi-level CNN's (ML-CNN's), could be employed at this step. A person skilled in the art would understand that other neural networks, deep learning techniques and artificial intelligent optimization models could also be employed for this task.

At each step, the ML-CNN takes as input a CT image volume sampled with a finer spatial resolution and a more restricted field of view, while maintaining the image matrix size constant. At the first resolution step, the raw CT image data was centered about its 3D midpoint and resampled to an isotropic voxel spacing of 4 millimeters and an image matrix of 64×64×64 voxels. At this spacing, the 256 mm field of view contained nearly the entire original image, and the aortic valve center was certain to be captured. This image matrix was passed to the top-level CNN, which predicted the 3D location of the aortic valve annulus centroid as output. This process was repeated with a pixel spacing of 2 mm then 1 mm. At the final step, the CNNs predicted the 3D location of the leaflet coaptation central point, and the 3D orientation of the aortic valve annulus viewed en face expressed as a 3D normal vector.

It is pertinent to note that in total, each model contains 34,426,037 trainable parameters, which were initialized randomly. The models were trained for 300 epochs and data augmentation included random variation in 3D translation by 10% of image field of view, 3D rotation by 40 degrees, and scale by 20% of the pixel spacing.

### Aortic Valve Calcium Segmentation

The task of determining if a voxel containing calcium is part of the aortic valve was solved using a segmentation algorithm. A neural network architecture was used that combined the block-level design of the ResNet model (9) with the overall structure of a U-net (10) and included multichannel geometric a priori. The proposed segmentation algorithm includes two technical aspects: (1) neural network design and (2) the use of image channels to integrate geometrical a priori information obtained from the landmarking stage of the algorithm. This combination resulted in a much deeper network referred to as a deep learning convolutional neural network (DL-CNN) (e.g. U-ResNet). A person skilled in the art would appreciate that other deep-learning and convolutional neural networks available in the art could also be employed for this task. Additional channels were included to provide structural information known a priori as input to the model. This model is referred to as multi-channel deep learning convolutional neural network (e.g. U-ResNet (MCU-Resnet)), also referred to as DL-CNN (defined as S. in the equation). The following channels were included:
1) the CT pixel data μ∈ [symbol] ^{MNO}, where the image matrix is of dimension M×N×O, and i, j, k are positional indices in the x, y, z directions respectively. The dynamic range of CT pixel data was set from −200 to 300 HU. The field of view of the input image volume was restricted to a region of interest surrounding the aortic root. The image matrix size was set to 32×128×128, thus capturing the entire aortic root while minimizing the amount of non-valvular calcium. In some embodiments, the image size matrix can be set to 16×64×64, 8×32×32, 4×16×16 or 2×4×4. The pixel and slice spacing of the original CT volume were retained.
2) a binary mask t∈{0,1}^{MNO} of all calcium based on thresholding where:

$${t}_{i j k} = \{ \begin{matrix}0 & {μ}_{i j k} < 130 HU \\ 1 & {μ}_{i j k} \geq 130 HU\end{matrix}$$

3) the 3D radial distance from the AV or Aortic Root axis r∈ [symbol] ^{MNO} where:

rᵢⱼₖ(v,\hat{n})=∥(xᵢⱼₖ−v)−\hat{n}((xᵢⱼₖ−v)·\hat{n})∥
4) the distance relative to the aortic valve or aortic root annular plane d∈ [symbol] ^{MNO} where:

dᵢⱼₖ(v,\hat{n})=(x_{i,j,k}−v)·\hat{n}

xᵢⱼₖ is the spatial position at the center of voxel i, j, k. v is the annulus centroid position. \hat{n} is the unit normal vector of the aortic annulus plane. The segmentation neural network Sₙₙ: [symbol] ^{4MNO}→{0,1}^{MNO} takes as input the 4 channels described above:

$$m = S ( μ , v , \hat{n} ) = {S}_{n n} ( [ \begin{matrix}μ \\ t ( μ ) \\ r ( v , \hat{n} ) \\ d ( v , \hat{n} )\end{matrix} ] )$$

The output mask m∈{0,1}^{MNO} is an array of the same shape as the input image, where the value of each voxel was 1 if the voxel was predicted to be AVC, and 0 otherwise.

Additional channels may be included to provide structural information known a priori as input to the model. Optionally, the projected out-of-plane 3D distance along the AV or aortic root axis relative to the AV or aortic root coaptation point could also be included.

It is pertinent to note that in total, the segmentation algorithm contained 15,920,769 trainable parameters, which corresponded to 183 MB of disk space. The model was trained over 300 epochs and data augmentation included 2D rotations by up to 40 degrees, and 2D translations by up to 10% of the image size in-plane only. The performance of the several neural network architectures were determined for instance, U-Net, MCU-Net, and proposed two-stage algorithm i.e. ML-CNN/DL-CNN (e.g. MCU-ResNet). All convolutions were configured to be 3D.

### Computing Environment

For the purposes of building and training neural networks, TensorFlow version 2.3.0 and Keras version 2.4.0 were used. Machine learning hyperparameters including the learning rate and batch size were optimized using a Bayesian algorithm in the platform Weights and Biases (11). For numerical manipulation and statistical analysis, NumPy version 1.19.5, SciPy version 1.5.4, and Pandas version 1.1.3 were used. Finally, Matplotlib version 3.3.2 was used for figure creation.

A person skilled in the art would understand that other alternative techniques such as MATLAB, IBM Watson Studio, Google Cloud AI Platform, Theano, PyTorch, OpenCV, Keras, Apache Spark, Amazon SageMaker, Google Cloud AutoML, RapidMiner, Azure Machine Learning Studio, and Anaconda could also be employed.

### Statistical Analysis

A thorough nine-fold cross-validation was applied to both the landmarking and segmentation components of the algorithm. As such, the scans were randomly split into nine equally sized groups. If a patient had multiple CT scans, the groups were adjusted so that all scans for that patient would belong to a single group. In total nine models were trained.

The performance of the feature landmarking CNN was evaluated using the 3D position error, which is defined as the Euclidian distance between the predicted and labeled feature position. The accuracy of the 3D orientation CNN was evaluated using the 3D angular error measured in degrees. The accuracy of the segmentation algorithm was quantified using a dice coefficient, the mean average error (MAE) and mean relative error (MRE), measured between the AVC score calculated from the predicted and labeled segmentation masks:

$${σ}_{MAE} = \frac{1}{N} {\sum }_{n = 0}^{N - 1} \vert {c}_{n} - {\tilde{c}}_{n} \vert$$

$${σ}_{MRE} = \frac{2}{N} {\sum }_{n = 0}^{N - 1} \frac{\vert {c}_{n} - {\tilde{c}}_{n} \vert}{\vert {c}_{n} + {\tilde{c}}_{n} \vert}$$

where there the sum is over N patients, cₙ is the predicted AVC score, and on is the labeled AVC score. In some instances, the performance of the feature landmarking CNN could be evaluated using the 3D orientation error by similar means.

To evaluate the dependence on the amount of AVC, the results were reported in the following ranges of AVC scores: 0-100 AU, 100-750 AU, 750-1400 AU, 1400-2050 AU and >2050 AU.

Additionally, the MAE and MRE were evaluated for the entire aortic valve as well as for its subregions. A Bland-Altman analysis was performed and a correlation analysis using the coefficient of determination (R2) between the labeled and predicted AVC scores was carried out as well. The relative and absolute interobserver difference was calculated, as well as repeated the Bland-Altman and correlation analyses for AVC scores between the fourth operator and each of three other operators. The AVC score quantification was also used to evaluate the diagnostic accuracy (area under the receiver operating characteristic curve [ROC AUC], sensitivity, specificity, positive and negative predictive value) to identify patients with severe AS based on well-accepted sex-specific thresholds (1300 AU in women and 2000 AU in men). Confidence intervals (CI) for sensitivity, specificity and accuracy are exact Clopper-Pearson confidence intervals and all other CI were calculated using bootstrapping with 10,000 resamples.

A person skilled in the art would appreciate that various other testing and validation techniques can be employed to determine accuracy of the algorithms.

### Discussion of Results

### Population

Patients from a single academic hospital were included in the training dataset and separated in two cohorts: (1) AS cohort and (2) non-AS cohort. The AS cohort comprised consecutive patients referred for a transcatheter or surgical aortic valve replacement and who underwent a clinically indicated CT calcium score between 2000 and 2021. The non-AS cohort was composed of consecutive patients who underwent a cardiac CT calcium score for assessment of coronary artery disease in 2020. Clinical and echocardiographic data were extracted from the clinical electronic medical record. Patients with a prior aortic valve replacement and Sievers type 0 bicuspid aortic valve were excluded from the database. Furthermore, patients without a CT scan or with CT images of poor quality due to truncation, beam hardening, and/or motion artifacts were also excluded. CT scans acquired at a tube voltage other than 120 kVp were excluded. Approval was obtained for the study from the Research Ethics Board at the institution. Patient consent was waived given the retrospective nature of this analysis.

The final database included a dataset of 845 CT scans, 726 CT scans in 708 patients with AS referred for aortic valve intervention and 119 CT scans in 119 control patients (non-AS cohort) who underwent a CT calcium scoring for the evaluation of coronary artery disease. The annotated scans were used to train the neural networks and nine-fold cross-validation was used to evaluate their performance. The clinical characteristics of the AS cohort are provided in Table 1. Briefly, mean age was 82±8 years, 44% were women, mean pressure gradient was 44±15 mm Hg and mean AVC score 3299±1808 AU. 436 (62%) patients were considered as having severe AS based on mean pressure gradient (≥40 mm HG) and 608 (86%) using AVC and sex-specific thresholds (1300 AU in women and 2000 AU in men). Most of the control patients 113 (95%) had minimal AVC (<100 AU).

TABLE 1
Clinical characteristics of the aortic stenosis cohort

| | Aortic stenosis |
|---|---|
| | cohort |
| | (n = 708 |
| | patients) |
| Characteristics | |
| Age | 82.2 ± 7.5 |
| Female sex - no. (%) | 310 (44%) |
| Height (cm) | 165.8 ± 12.2 |
| Weight (kg) | 77.5 ± 18.8 |
| Body surface area (m²) | 1.87 ± 0.26 |
| Comorbidities | |
| Diabetes mellitus - no. (%) | 233 (33%) |
| Hypertension - no. (%) | 564 (80%) |
| Coronary artery disease - no. (%)* | |
| Zero | 246 (35%) |
| One | 150 (21%) |
| Two | 136 (19%) |
| Three | 156 (22%) |
| Heart Failure Functional Class - no. (%)* | |
| NYHA I/II | 257 (36%) |
| NYHA III/IV | 435 (61%) |
| Echocardiographic assessment | |
| Left ventricular ejection fraction (%) | 54.7 ± 11.2 |
| Aortic valve area (cm²) | 0.75 ± 0.18 |
| Indexed aortic valve area (cm²/m²) | 0.39 ± 0.09 |
| Mean pressure gradient (mmHg) | 44.4 ± 14.5 |
| Moderate or worse aortic regurgitation - no. (%) | 257 (36%) |
| Moderate or worse mitral regurgitation - no. (%) | 269 (38%) |
| Moderate or worse tricuspid regurgitation - no. (%) | 209 (30%) |
| CT aortic valve calcium score (AU) | |
| Total | 3299 ± 1808 |
| Right coronary cusp | 830 ± 563 |
| Left coronary cusp | 844 ± 524 |
| Non coronary cusp | 1099 ± 624 |
| Left ventricular outflow tract | 526 ± 671 |

*Coronary artery anatomy was not available in 20 patients (3%) and NYHA class was not available in 16 patients (2%).

In some embodiments, the algorithm could be further trained using patient data with and without pre-diagnosed confirmed AS. This would help the algorithm to differentiate between a patient that shows severe AVC versus patients that display negligible AVC.

### Aortic Root Anatomical Feature Landmarking

During cross-validation, the multi-scale anatomical feature landmarking achieved a mean 3D position error of 2.7 mm (95% Cl 2.6 mm to 2.8 mm) and a mean angular error of 10.5° (95% Cl 10.1° to) 10.9°. For AVC segmentation using the U-Net, MCU-Net and MCU-ResNet models, the mean Dice coefficients were respectively 0.70 (95% confidence interval [CI] 0.68-0.72), 0.76 (95% Cl 0.74-0.78), 0.84 (95% CI 0.82-0.86), the mean average errors in AVC score were 920 (95% Cl 840-1004), 491 (95% CI 444-541), 136 (95% Cl 116-158), and the mean relative errors were 28.9% (95% Cl 26.8%-31.2%), 17.8% (95% Cl 16.3%-19.6%), 5.5% (95% Cl 4.5%-6.7%). Whereas the U-Net and MCU-Net often misclassified coronary artery calcium as AVC, the MCU-ResNet performed significantly better. For fully automated diagnosis of severe AS, the MCU-ResNet reached a sensitivity 96.9% (95% Cl 95.0%-98.2%) and a specificity of 96.0% (95% Cl 93.2%-97.9%).

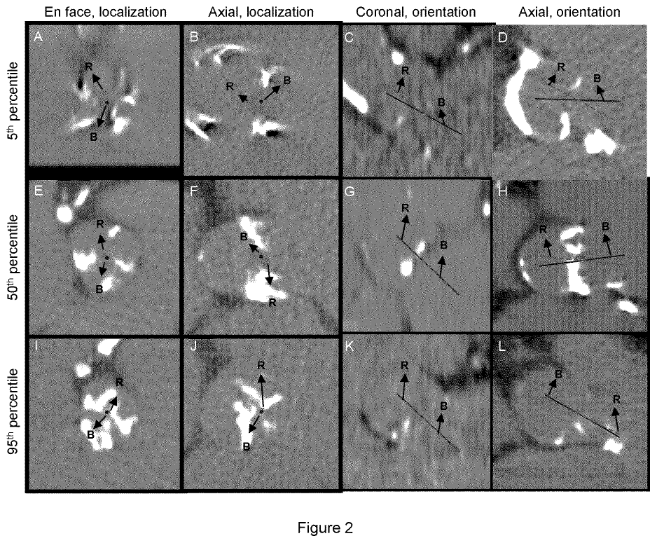

FIG. 2 shows sample CT images with manually annotated and multi level convolutional neural network (ML-CNN) predicted aortic valve landmarks. Manually annotated anatomical features (leaflet coaptation point [circles/crosses] and aortic root longitudinal axis [lines]) are presented in blue (B) and model predictions in red (R). The top, middle, and bottom rows show illustrative scans of patients who were in the 5^{th}, 50^{th}, and 95^{th} percentiles in model accuracy. In summary, illustrative examples of the predicted position of the leaflet coaptation point and aortic root orientation of three patients in the 5^{th}, 50^{th} and 95^{th} model accuracy compared to those manually annotated. Importantly, the accuracy of the algorithm was not influenced by the amount of aortic valve calcification.

### Aortic Valve Calcium Segmentation (Stage 2)—Total AVC Score

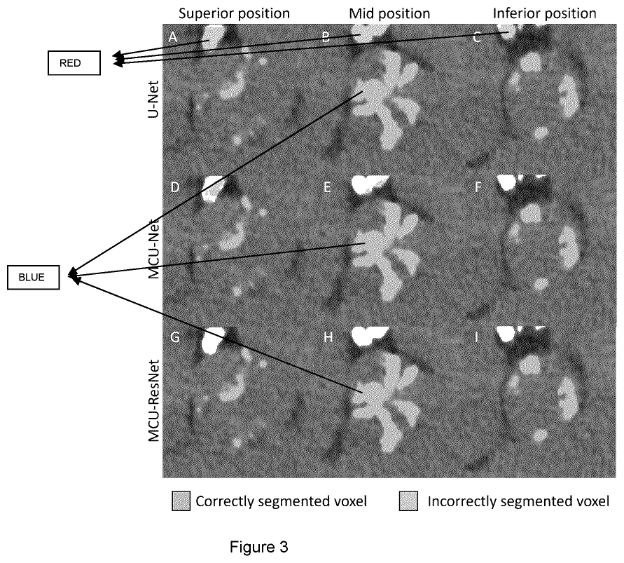

The segmentation models were trained on the dataset using nine-fold cross-validation. For this part of the evaluation, the labeled position and the orientation of the aortic valve were used as input to the multi-channel (MC) algorithms. This was done to evaluate the performance of the segmentation stage of the algorithm in isolation. FIG. 3 provides sample CT images with overlay of segmented aortic calcium mask using three algorithms. Concordant voxels between the labelled segmentation and the model-predicted segmentation are blue, and discordant voxels are shown in red. FIG. 3 mainly illustrates the segmentation results from CT scans with corresponding manual and automated AVC segmentation for each algorithm.

It is pertinent to note that the AVC was correctly identified for all three algorithms; however, coronary calcium was mis-identified as AVC by the U-Net and MCU-Net algorithms. The MCU-ResNet algorithm correctly identified coronary calcium as such. The mean Dice coefficients for the U-Net, MCU-Net and MCU-ResNet were respectively 0.70 (95% Cl 0.68-0.72), 0.76 (95% Cl 0.74-0.78), 0.84 (95% Cl 0.82-0.86); the Dice coefficients were significantly different with p<0.001 when compared as pairs.

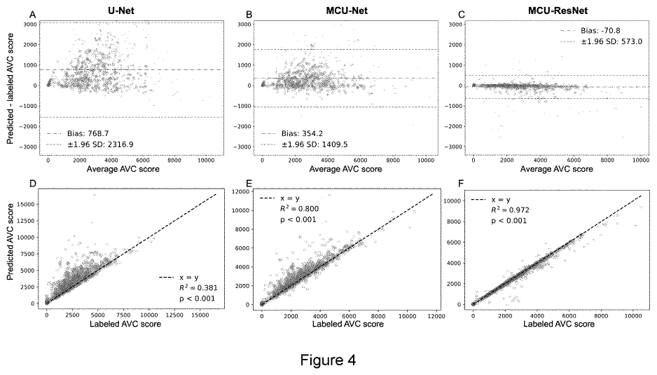

Total AVC score accuracy: The first row of Table 2 provides the overall accuracy of each segmentation models based on the MAE and MRE for the AVC score. The MCU-ResNet resulted in the lowest absolute and relative errors compared to other models (p<0.001). As shown in Bland-Altman analysis (FIG. 4), the U-Net and MCU-Net resulted in a systematic overestimation of the calcium score (bias of 769 and 354), while the MCU-ResNet did not (bias-71). FIG. 4 provides Bland-Altman plots (A-C) and correlation plots (D-F) comparing the labeled to the model-predicted AVC scores for corresponding patients. The proposed DL-CNN also resulted in the highest correlation with the labeled AVC score (R²=0.972) compared to the U-Net (R²=0.381), and MCU-Net (R²=0.800).

TABLE 2 provides Mean absolute and relative error in total and region-specific aortic valve calcium (AVC) scores for the three segmentation models (Stage 2 only). MAE: mean absolute error; MRE: mean relative error; CI: confidence interval; RCC: right coronary cusp; LCC: left coronary cusp; NCC: non-coronary cusp; LVOT: left ventricular outflow tract.

| | Mean absolute error (95% CI) | | | | | | Mean relative error - % (95% CI) | | | | | |
|---|---|---|---|---|---|---|---|---|---|---|---|---|
| Region | U-Net | | MCU-Net | | MCU-ResNet | | U-Net | | MCU-Net | | MCU-ResNet | |
| Total | 920 | (840-1004) | 491 | (444-541) | 136 | (116-158) | 28.9 | (26.8-31.2) | 17.8 | (16.3-19.6) | 5.5 | (4.5-6.7) |
| RCC | 352 | (316-391) | 144 | (128-164) | 37 | (30-45) | 39.1 | (35.9-42.4) | 21.9 | (19.2-24.6) | 7.1 | (5.6-8.7) |
| LCC | 443 | (405-480) | 294 | (268-322) | 41 | (34-48) | 44.1 | (41.1-47.2) | 33.8 | (31.3-36.6) | 6.3 | (5.1-7.7) |
| NCC | 141 | (121-163) | 99 | (86-113) | 52 | (42-62) | 14.7 | (13.0-16.4) | 10.9 | (9.6-12.6) | 6.4 | (5.0-8.1) |
| LVOT | 81 | (69-96) | 64 | (26-44) | 23 | (17-31) | 50.5 | (46.5-54.7) | 19.2 | (16.6-21.9) | 13.4 | (11.3-15.8) |

Regional AVC Score accuracy: Table 2 provides the regional error in AVC score of each segmentation models. The AVC score was least accurate in the right and left coronary cusps (RCC and LCC) for the U-Net and MCU-Net and most accurate in the non-coronary cusp (NCC). However, for the MCU-ResNet model, the MAE and MRE were not significantly different between the three coronary cusps. The MRE of the MCU-ResNet was higher in the left ventricular outflow tract region compared to the coronary cusps.

AVC score accuracy stratified by quantity of calcification: Table 3 presents the AVC score error for each algorithm stratified based on the amount of calcium for each subject. The MAE is a useful metric in the lower scores and the MRE is useful in the higher scores. We note that the scans with an AVC score less than 100 resulted in a large relative error (MRE) but a low absolute error (MAE). In the range of 100 to 2050, the MAE does not vary significantly for all models studied and the MRE decreases as the quantity of calcium increases. For the MCU-ResNet model, the MRE remains in a similar range for AVC scores over 2050 but the MAE higher in that range. In all AVC score ranges, the MCU-ResNet has a lower MAE and MRE than either of the MCU-Net or the U-Net algorithm.

Table 3 provides the mean absolute and relative error in aortic valve calcium (AVC) scores stratified by quantity of aortic calcification for each segmentation model (Stage 2 only).

| | Mean absolute error (95% CI) | | | | | | Mean relative error - % (95% CI) | | | | | |
|---|---|---|---|---|---|---|---|---|---|---|---|---|
| AVC score range | U-Net | | MCU-Net | | MCU-ResNet | | U-Net | | MCU-Net | | MCU-ResNet | |
| 0 to 100 | 129 | (50-227) | 28 | (12-50) | 6.0 | (2.7-9.6) | 126 | (100-151) | 85 | (57-118) | 55 | (31-82) |
| 100 to 750 | 730 | (385-1118) | 485 | (226-777) | 63 | (29-100) | 65 | (48-85) | 48 | (30-66) | 16 | (7-27) |
| 750 to 1400 | 946 | (711-1207) | 539 | (389-670) | 51 | (33-76) | 47 | (39-54) | 32 | (26-38) | 5.4 | (3.1-8.8) |
| 1400 to 2050 | 882 | (724-1973) | 434 | (346-536) | 55 | (35-83) | 34 | (29-39) | 20 | (16-23) | 3.3 | (2.0-5.4) |
| ≥2050 | 936 | (828-1057) | 497 | (436-565) | 182 | (152-213) | 22 | (20-24) | 13 | (12-14) | 5.9 | (4.5-7.3) |

The landmarking and segmentation algorithms were combined, i.e., the aortic valve position and orientation predicted by the landmarking algorithm was used to center the field of view available to the segmentation algorithms, as well as to compute the geometrical channels for the MCU-Net and MCU-ResNet algorithms. These combined algorithms were fully automated. The MAE was 1145 (95% Cl 1052-1254), 628 (95% Cl 568-695), and 247 (95% Cl 217-281), for the U-Net, MCU-Net, and MCU-ResNet respectively. The MRE was 34% (95% Cl 32-37%), 22% (95% Cl 20-24), and 9.7% (95% CI 8.1-11.3).

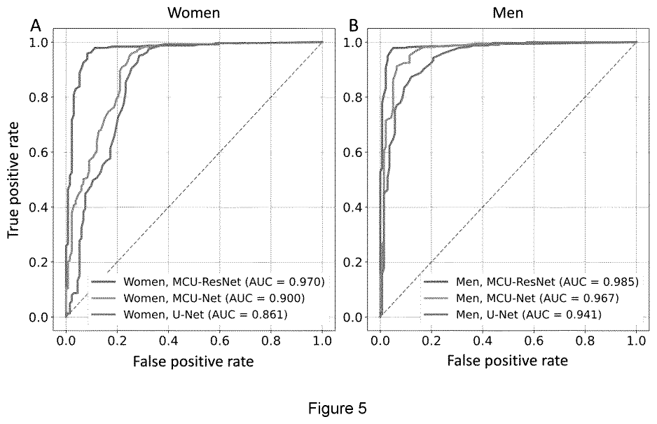

The performance of each algorithm for the diagnosis of severe aortic valve calcification, a clinically relevant metric that is used as an indication for aortic valve intervention based on sex specific AVC thresholds (1300 in women and 2000 in men). The diagnostic performance metrics are presented in Table 4. The ROC analysis is presented in FIG. 5. FIG. 5 provides receiver-operator characteristic curves for the diagnosis of severe aortic valve calcification using the three models (combined stages 1 and 2). Overall, the MCU-ResNet algorithm outperformed the other models for the diagnosis of severe aortic valve calcification.

Table 4 provides diagnostic performance for fully automated AVC scoring using the three models studied (combined stages 1 and 2):

| Diagnostic performance | U-Net | | MCU-Net | | MCU-ResNet | |
|---|---|---|---|---|---|---|
| Sensitivity | 98.2% | (96.6%-99.1%) | 97.79% | (96.2%-98.8%) | 96.9% | (95.0%-98.2%) |
| Specificity | 63.6% | (57.9%-69.0%) | 77.15% | (72.00%- | 96.0% | (93.2%-97.9%) |
| Positive Likelihood Ratio | 2.7 | (2.3-3.1) | 4.3 | (3.5-5.3) | 24 | (14-42) |
| Negative Likelihood Ratio | 0.03 | (0.02-0.05) | 0.03 | (0.02-0.05) | 0.03 | (0.02-0.05) |
| Accuracy | 85.8% | (83.3%-88.1%) | 90.4% | (88.2%-92.3%) | 96.6% | (95.1%-97.7%) |

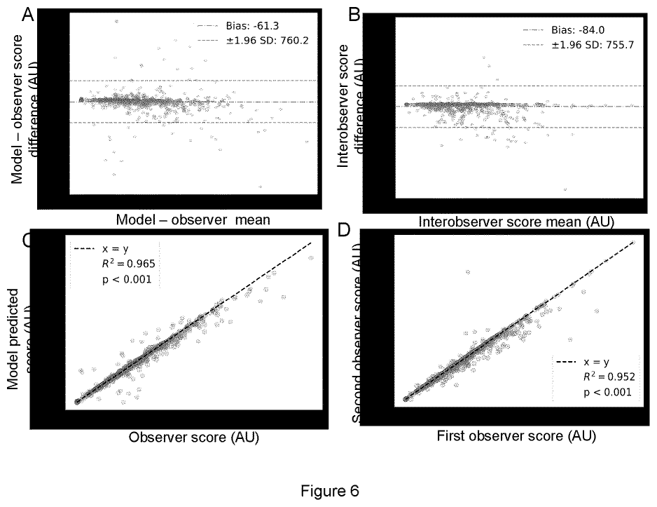

The correlation between AVC calcium score manually measured and the AVC score predicted by the segmentation algorithm was excellent (R2=0.965, p<0.001). As shown in the Bland-Altman analysis, there was no trend toward systematic under- or overestimation (FIGS. 6A and C). FIG. 6 shows a comparison of total aortic valve calcification (AVC) measured using the deep learning convolutional neural network (DL-CNN) algorithm and manually segmented AVC (A and C) and comparison of manually segmented AVC measured by different observers (B and D). Furthermore, Bland-Altman plots (A and B) and linear correlation analysis (C and D) using the coefficient of determination (R²) are presented as well.

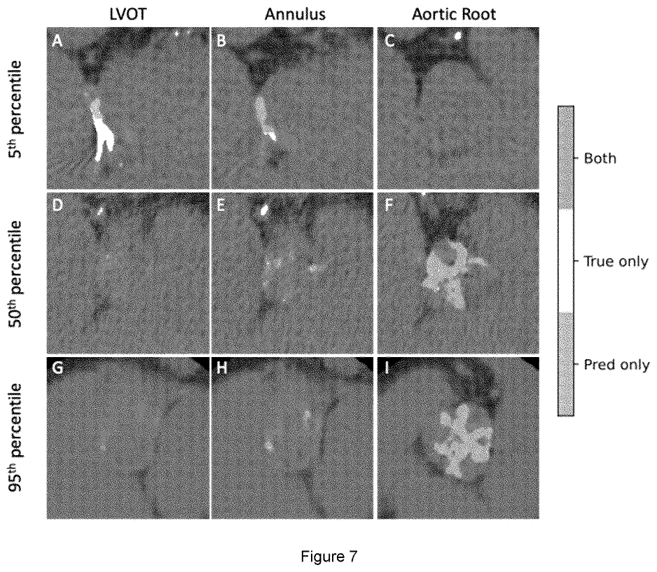

FIG. 7 shows sample CT images with manually annotated and deep learning convolutional neural network (DL-CNN) algorithm predicted aortic valve calcium masks overlay. The top, middle, and bottom rows show scans in the 5th, 50th, and 95th percentiles of model accuracy. The left, middle, and right columns respectively show slices from the left ventricular outflow tract (LVOT), the aortic valve annulus, and the aortic root. Segments of the images highlighted in blue, yellow, and red correspond respectively to AVC as determined by both the model and human annotators, only the human annotators, and only the algorithm. All slices are shown en face. The figure also shows the Proof of Concept Test AUCs as conducted using an initial 1,437 patient cohort. In summary, FIG. 7 illustrates the segmentation results from three sample CT scans with corresponding manual and automated AVC segmentation at three accuracy levels of the predicted aortic valve calcium score.

The correlation analysis between patients' clinical characteristics and the segmentation algorithm performance is presented in Table 6. A negligible degree of correlation was observed for most clinical characteristics. A low degree of correlation was observed between the MAE and the AVC score. No such correlation was observed between the MRE and the AVC score value.

TABLE 6
Correlation between the algorithm performance
metrics and clinical characteristics.

| | Correlation | | Correlation | |
|---|---|---|---|---|
| | with mean | | with mean | |
| | absolute error | | relative error | |
| | Pearson r | p-value | Pearson r | p-value |
| Characteristic | | | | |
| Age | 0.03 | 0.47 | 0.02 | 0.60 |
| Female sex - no. (%) | −0.04 | 0.36 | 0.04 | 0.33 |
| Height (cm) | 0.03 | 0.48 | −0.07 | 0.06 |
| Weight (kg) | 0.00 | 0.90 | −0.08 | 0.04 |
| Body surface area (m²) | 0.00 | 0.96 | −0.07 | 0.16 |
| Comorbidities | | | | |
| Diabetes mellitus | 0.02 | 0.63 | −0.01 | 0.85 |
| Hypertension | 0.00 | 0.93 | −0.03 | 0.47 |
| Coronary artery disease | 0.01 | 0.83 | 0.00 | 0.99 |
| (no. of diseased vessels) | | | | |
| Heart Failure Functional Class | 0.00 | 0.94 | 0.04 | 0.28 |
| Echocardiographic assessment | | | | |
| Left ventricular ejection | 0.00 | 0.94 | −0.01 | 0.83 |
| fraction | | | | |
| Aortic valve area | 0.05 | 0.17 | 0.06 | 0.15 |
| Indexed aortic valve area | 0.01 | 0.81 | 0.10 | 0.06 |
| Mean pressure gradient | 0.03 | 0.42 | −0.09 | 0.02 |
| Aortic regurgitation | 0.10 | 0.008 | 0.11 | 0.01 |
| Mitral regurgitation | 0.04 | 0.33 | 0.04 | 0.26 |
| Tricuspid regurgitation | 0.00 | 0.99 | 0.05 | 0.24 |
| Aortic valve calcium score | | | | |
| Total | 0.32 | <0.001 | −0.05 | 0.21 |
| Right coronary cusp | 0.26 | <0.001 | −0.05 | 0.16 |
| Left coronary cusp | 0.26 | <0.001 | −0.04 | 0.35 |
| Non coronary cusp | 0.21 | <0.001 | −0.08 | 0.05 |
| Left ventricular outflow tract | 0.28 | <0.001 | 0.04 | 0.28 |

The difference between the AI-model and human interobserver variability was compared (FIGS. 6 B and D). Numerical differences between the algorithm-predicted and the manually annotated AVC score and differences between interobserver are presented in Table 7. Focusing on patients with clinically meaningful AVC scores (>100 AU), the segmentation algorithm absolute error (164 AU, 95% Cl 142 AU to 191 AU) approached but did not reach the absolute interobserver difference (126 AU, 95% CI 100 AU to 154 AU; p=0.02). However, for the same range of AVC score, the relative error between the segmentation algorithm and human annotated score was not significantly different (6.5%, 95% CI 5.4% to 7.7%) from the relative interobserver difference (5.2%, 95% Cl 3.9% to 6.8%; p=0.12). The error was stratified based on the overall AVC score (Table 8). The MAE increased in patients with higher AVC scores, but the MRE remained stable for higher AVC scores.

TABLE 7
Absolute and relative differences in total and region-specific aortic valve
calcium (AVC) scores between observers and the proposed algorithm.

| | Absolute comparison - AU (95% CI) | | | | | Relative comparison - % (95% CI) | | | | |
|---|---|---|---|---|---|---|---|---|---|---|
| | | | Interobserver | | | | | Interobserver | | |
| Region | Algorithm MAE | | absolute difference | | p-value | Algorithm MRE | | relative difference | | p-value |
| Total | 164 | (142-191) | 126 | (100-154) | 0.02 | 6.5 | (5.4-7.7) | 5.2 | (3.9-6.8) | 0.12 |
| RCC | 37.5 | (30.1-46.0) | 17.9 | (10.1-27.6) | <0.001 | 7.2 | (5.7-8.9) | 2.7 | (1.5-4.0) | <0.001 |
| LCC | 39.9 | (33.1-48.3) | 15.2 | (9.8-22.3) | <0.001 | 6.3 | (5.0-7.7) | 2.9 | (1.7-4.3) | <0.001 |
| NCC | 49.2 | (40.1-58.7) | 17.4 | (10.2-26.9) | <0.001 | 6.3 | (4.9-8.0) | 2.7 | (1.5-4.3) | <0.001 |
| LVOT | 58.9 | (47.4-71.1) | 83.8 | (69.0-101.3) | 0.005 | 24.0 | (21.1-27.0) | 30.6 | (26.7-35.0) | 0.003 |

MAE: mean absolute error; MRE: mean relative error; CI: confidence interval; RCC: right coronary cusp; LCC: left coronary cusp; NCC: non-coronary cusp; LVOT: left ventricular outflow tract.

TABLE 8
Absolute and relative differences in total and region-specific aortic valve calcium (AVC) scores
between observers and the proposed algorithm stratified by degree of aortic valve calcification.

| | | Mean absolute difference | | | | Mean relative difference | | | |
|---|---|---|---|---|---|---|---|---|---|
| | | stratified by total AVC score (AU) | | | | stratified by total AVC score (%) | | | |
| Comparison | Region | Overall | 100-1000 | 1000-2000 | >2000 | Overall | 100-1000 | 1000-2000 | >2000 |
| Algorithm predicted and | Total | 164.0 | 87.4 | 68.8 | 208.8 | 6.5 | 12.1 | 5.0 | 6.5 |
| human annotated AVC score | RCC | 37.5 | 8.9 | 14.1 | 49.6 | 7.2 | 21.0 | 4.7 | 6.6 |
| | LCC | 39.9 | 17.4 | 20.2 | 50.0 | 6.3 | 14.9 | 6.6 | 5.2 |
| | NCC | 49.2 | 14.4 | 23.7 | 62.8 | 5.3 | 7.7 | 5.4 | 6.5 |
| | LVOT | 58.8 | 53.6 | 25.3 | 72.3 | 24.0 | 45.5 | 24.3 | 21.6 |
| Interobserver difference | Total | 125.8 | 39.9 | 63.4 | 159.1 | 5.1 | 9.4 | 4.7 | 4.9 |
| in AVC score | RCC | 17.9 | 10.2 | 6.6 | 23.1 | 2.7 | 4.3 | 2.3 | 2.6 |
| | LCC | 15.2 | 9.4 | 11.1 | 17.5 | 2.9 | 7.5 | 3.0 | 2.4 |
| | NCC | 17.4 | 6.0 | 9.8 | 21.6 | 2.7 | 6.3 | 2.5 | 2.4 |
| | LVOT | 83.8 | 14.5 | 43.9 | 106.8 | 30.6 | 21.4 | 31.2 | 31.4 |

RCC: right coronary cusp; LCC: left coronary cusp; NCC: non-coronary cusp; LVOT: left ventricular outflow tract.

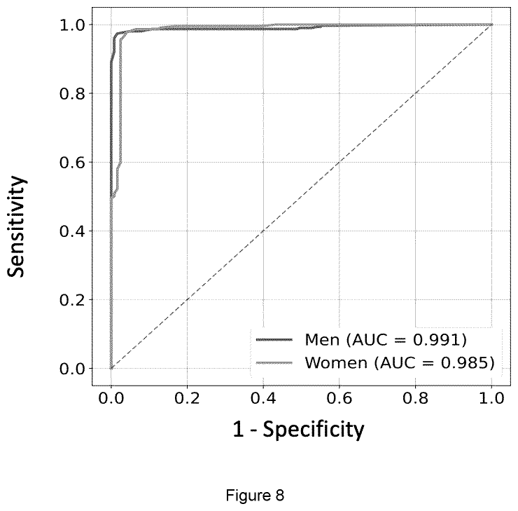

The model achieved a high accuracy for the diagnosis of severe AS (96.3%, 95% Cl 94.8% to 97.5%). The sensitivity and specificity were 95.0% (95% Cl 92.7% to 96.6%) and 99.0% (95% Cl 97.0% to 99.8%) and the positive and negative predictive values 99.4% (95% Cl 98.3% to 99.8%) and 91.2% (95% Cl 87.8% to 93.7%) respectively. Similar accuracy was observed in men and women (area under the ROC curves 0.991 (95% Cl 0.983 to 0.997) and 0.985 (95% Cl 0.973 to 0.996) respectively). FIG. 8 presents the ROC curves for the identification of patients with severe AS based on sex specific AVC thresholds in men and women. In summary, FIG. 8 shows receiver-operator characteristics curves for severity classification stratified by patient sex.

### Aortic Valve Calcium Segmentation—Regional Analysis

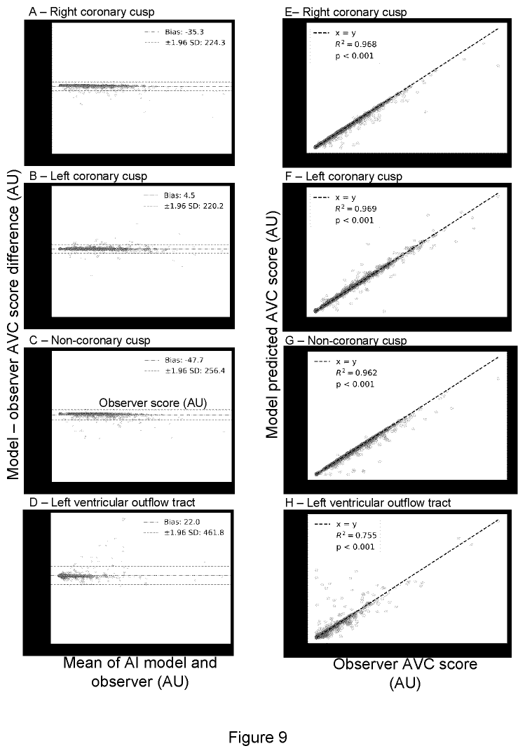

Correlations between AVC calcium score manually measured and the AI-predicted AVC score in four regions (right, left, and non-coronary cusp and left ventricular outflow tract) are presented in FIG. 9. FIG. 9 shows Bland-Altman plots (A to D) and correlation analysis (E to H) of the proposed ML-CNN and DL-CNN algorithm for automated AVC scoring in the four anatomical regions of the aortic root: the right coronary cusp (A and E), the left coronary cusp (B and F), the non-coronary cusp (C and G), and the left ventricular outflow tract (D and H). Overall correlations were high but lower for the LVOT region (E to H). There was again no trend toward over- or underestimation of AVC for each of these regions.

Table 7 and 8 present the MAE and MRE for each region. Generally, the MAE and MRE were similar for each of the three aortic valve cusps but, the error was higher in the LVOT region. A higher interobserver difference between human annotators was also observed in the LVOT compared to the three aortic cusps.

The algorithm error in the three cusps was significantly higher than the interobserver difference in the three aortic cusps (p<0.001). However, the algorithm significantly outperformed the interobserver difference in the LVOT region (p=0.005 for the absolute difference, p=0.003 for the relative difference).

The results of this study can be summarized as follows:
1. The multi-scale system of regression CNN for aortic root anatomical feature landmarking was adequately accurate to serve as input to the segmentation algorithm.
2. The multi-scale system of regression CNN for aortic root anatomical feature landmarking was able localize the aortic valve on average within 2.7 mm, and the orientation within 10.5°.
3. The proposed MCU-ResNet outperformed the U-Net and MCU-Net architectures in terms of segmentation accuracy based on F1 score and on AVC score MAE and MRE.
4. The MCU-ResNet performed best at differentiating coronary calcium from AVC; its accuracy did not depend on the coronary cusp. The U-Net and MCU-Net segmentation model had a worse performance in the RCC and LCC and performed best in the NCC. These models erroneously segmented coronary calcium as AVC, which explains the worse performance in the RCC and LCC.
5. The MCU-ResNet did not systematically over- or under-estimate the AVC score. However, both other models overestimation of the AVC score.
6. The MCU-ResNet model outperformed the other models regardless of the amount of calcium present on the valve
7. For the fully automated diagnosis of severe aortic valve calcification, the MCU-ResNet model performance was excellent and was not significantly biased with respect the patient sex. The other two models had worse diagnostic performance in female patients
8. The proposed hybrid 3D U-Net and ResNet architecture with multichannel geometric a priori achieved an absolute and relative error level that approached human level accuracy for clinically relevant scans with a total AVC greater than 100 AU. Both human annotators and the algorithm were most accurate for calcium contained within the aortic valve leaflets and least accurate for LVOT calcium.
9. The combination of the anatomical feature landmarking CNN and segmentation algorithms to identify patients with severe AS based on well accepted sex-specific thresholds resulted in a high diagnostic performance. The performance was similar for both men and women.
10. The segmentation algorithm performance was not significantly biased with respect to the clinical characteristics studied including sex, age, weight, height, and comorbidities.

The current study used an automated landmarking algorithm (ML-CNN) for the aortic valve and a hybrid 3D U-Net and ResNet architecture (DL-CNN) with multichannel geometric a priori for segmentation (MCU-ResNet). A comparison of the method with a standard 3D U-Net with (MCU-Net) and without (U-Net) multichannel geometric a priori was also conducted. Using the approach described herein, the restricted volume of the aortic valve was taken advantage of by first finding its location and then restricting the field of view to the surrounding region, thus simplifying the segmentation task. The multichannel approach used here is also novel and allowed to provide a priori information obtained from the landmarking algorithm as input to the segmentation algorithm. Based on a sample size two times larger and including a greater proportion of patients with AS, the proposed algorithm achieved an excellent correlation with annotated measurements with no trend toward a systematic under- or over estimation. The results approached human accuracy and reproducibility. Diagnostic accuracy for severe AS was excellent although it should be acknowledged that AVC is a continuum and that thresholds were used for statistical purpose. Importantly, a similar accuracy was observed for men and women. Although an excellent accuracy was achieved for the localization of the aortic valve and quantification of the degree of AVC, given the absence of intravenous contrast agent, anatomical features relied on the variable presence of aortic calcification and the low-contrast difference between blood and cardiac structures explains the slightly lower accuracy achieved in this study compared to that previously published for contrast-enhanced CT.

The AI-model performed well for all three cusps, but a lower accuracy was noted in the LVOT region. This lower accuracy was observed for both the algorithm and human annotators highlighting challenges posed by this region and the continuity of calcification between the aortic valve and the mitral valve annulus.

### Study Limitations

Strengths and limitations of the present study deserve comments. The number of patients included in this study remained relatively limited and most patients with AS were referred for an intervention. A larger dataset including patients with mild or moderate aortic stenosis may further improve the accuracy of the algorithm. Nevertheless, consecutive CT scans of AS patients were collected, and enriched our population with controls mostly free of aortic valve calcification to challenge the model. Despite a lower contrast with adjacent structure and a lower spatial resolution, analysis was performed on non-contrast CT. However, AVC measurements, thresholds and Agatston scoring system have only been validated in this setting. A limiting distance of 10 mm below the aortic valve annulus was arbitrarily chosen as criterion to exclude mitral annular calcium. However, there is no validated threshold, and a value was required to standardize the manual segmentation and measurements performed by the AI model.

### Clinical Implications

This technique may have potential clinical implications. Automatic AVC measurement and our algorithm could be incorporated into CT image analysis software and may improve efficiency and accuracy. In parallel, to continuing improving its accuracy, the model will be tested in the clinical setting, and the potential gain in time, accuracy and reproducibility for both experienced and less experienced operators will be evaluated. Models such as the one presented in this study may also facilitate the greater dissemination of AVC in clinical practice. The ability to divide the aortic valve automatically and accurately into regions may portend important pathophysiological implications regarding the natural history of AVC progression and the association between AVC and hemodynamic progression. Finally, AVC scoring has been shown to be predictive of periprocedural complications in patients undergoing transcatheter aortic valve implantation (TAVI). Calcification extending into the LVOT has been shown to predict the need for pacemaker implantation (22) and paravalvular leak after TAVI (23). It remains to be established whether total and segmental AVC measurements performed using machine learning ideally in addition with other information contained in the CT scan will improve the currently crude predictions of TAVI complications.

The landmarking stage of the algorithm achieved a 3D position error of 2.7 mm (95% Cl 2.6 mm to 2.8 mm) and a mean angular error of 10.5° (95% Cl 10.1° to) 10.9°. AVC measurement by the segmentation algorithm was highly correlated with manually measured AVC (R2=0.965, p<0.001) without a trend toward systematic under- or overestimation, and provided an accuracy of 96% for the diagnosis of severe AS. Mean absolute and relative errors were 164 AU (95% Cl 142-191) and 6.5% (95% Cl 5.4-7.7) respectively and approached the human interobserver difference (126 AU [95% Cl 100-154], p=0.02; 5.2% [95% Cl 3.9-6.8], p=0.12).

Hence, the proposed two-stage algorithm for automated segmentation of AVC approached human level accuracy for AVC scoring. A high diagnostic performance was achieved for the diagnosis of severe AS. In this large cohort of AS and control patients, an algorithm for both landmarking the aortic valve and measuring the total and segmental degree of AVC was developed and validated using gated non-contrast CT. The proposed two-stage algorithm for automated segmentation of AVC approached human level accuracy for AVC scoring based on convolutional neural networks and offered a high diagnostic performance for the diagnosis of severe AS using well validated sex-specific AVC score thresholds. Accuracy of the algorithm will continue to improve as larger numbers of CT will be incorporated and future research will test its clinical additional value in term of accuracy, reproducibility, gain of time and prediction of outcomes.

### Potential Applications

The quantification score could be helpful in determining if medical intervention is necessary. If a high AVC score is determined which indicates sever AS, perhaps, the CT scans of that patient could be monitored at regular intervals using the multi stage CNN proposed herein. In fact, after the algorithm determines a high AVC score, the algorithm could be trained to provide medical intervention recommendations. The scan and the score could be seen by a medical professional and further diagnosis could be carried out if necessary.

The proposed deep-learning algorithm could be simply downloaded on a computer-disk or some sort of readable medium and the disk can be used on a computer or similar device to localize and quantify AVC. Alternatively, the algorithm could be used on a computing system which is fed with CT scan images or any medical imaged to run the calculations and/or localize an isotropic point.

In some embodiments, the algorithm could be installed on a CT scanner or any imaging device such that the algorithm localizes the aortic valve and quantifies calcification on any new scan/image captured by the CT scanner or imaging device and calculates the AVC score instantaneously. Alternatively, an artificially intelligent device could be trained on using the algorithm to quantify calcification.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### REFERENCES

1. Messika-Zeitoun D., Aubry M-C., Detaint D., et al. Evaluation and Clinical Implications of Aortic Valve Calcification Measured by Electron-Beam Computed Tomography. Circulation 2004; 110 (3): 356-62. Doi: 10.1161/01.CIR.0000135469.82545.D0.
2. Vahanian A., Beyersdorf F., Praz F., et al. 2021 ESC/EACTS Guidelines for the management of valvular heart disease: Developed by the Task Force for the management of valvular heart disease of the European Society of Cardiology (ESC) and the European Association for Cardio-Thoracic Surgery (EACTS). Rev Esp Cardiol Engl Ed 2022; 75 (6): 524. Doi: 10.1016/j.rec.2022.05.006.
3. Otto C M., Nishimura R A., Bonow R O., et al. 2020 ACC/AHA Guideline for the Management of Patients With Valvular Heart Disease: A Report of the American College of Cardiology/American Heart Association Joint Committee on Clinical Practice Guidelines. Circulation 2021; 143(5). Doi: 10.1161/CIR.0000000000000923.
4. Clavel M-A., Messika-Zeitoun D., Pibarot P., et al. The Complex Nature of Discordant Severe Calcified Aortic Valve Disease Grading. J Am Coll Cardiol 2013; 62(24): 2329-38. Doi: 10.1016/j.jacc.2013.08.1621.
5. Pawade T., Sheth T., Guzzetti E., Dweck M R., Clavel M-A. Why and How to Measure Aortic Valve Calcification in Patients With Aortic Stenosis. JACC Cardiovasc Imaging 2019; 12(9): 1835-48. Doi: 10.1016/j.jcmg.2019.01.045.
6. Lauzier P T., Avram R., Dey D., Slomka P., Afilalo J., Chow B J W. The Evolving Role of Artificial Intelligence in Cardiac Image Analysis. Can J Cardiol 2021: S0828282X21007510. Doi: 10.1016/j.cjca.2021.09.030.
7. Agatston A S., Janowitz W R., Hildner F J., Zusmer N R., Viamonte M., Detrano R. Quantification of coronary artery calcium using ultrafast computed tomography. J Am Coll Cardiol 1990; 15(4): 827-32. Doi: 10.1016/0735-1097(90) 90282-T.
8. Theriault-Lauzier P., Alsosaimi H., Mousavi N., et al. Recursive multiresolution convolutional neural networks for 3D aortic valve annulus planimetry. Int J Comput Assist Radiol Surg 2020; 15(4): 577-88. Doi: 10.1007/s11548-020-02131-0.
9. Hara K., Kataoka H., Satoh Y. Can Spatiotemporal 3D CNNs Retrace the History of 2D CNNs and ImageNet? CoRR 2017;abs/1711.09577.
10. Ronneberger O., Fischer P., Brox T. U-Net: Convolutional Networks for Biomedical Image Segmentation. CoRR 2015;abs/1505.04597.
11. Biewald L. Experiment Tracking with Weights and Biases 2020.
12. Baumgartner H., Hung J., Bermejo J., et al. Recommendations on the Echocardiographic Assessment of Aortic Valve Stenosis: A Focused Update from the European Association of Cardiovascular Imaging and the American Society of Echocardiography. J Am Soc Echocardiogr 2017; 30(4): 372-92. Doi: 10.1016/j.echo.2017.02.009.
13. Pawade T., Clavel M-A., Tribouilloy C., et al. Computed Tomography Aortic Valve Calcium Scoring in Patients With Aortic Stenosis. Circ Cardiovasc Imaging 2018; 11(3): e007146. Doi: 10.1161/CIRCIMAGING. 117.007146.
14. Išgum I., Rutten A., Prokop M., van Ginneken B. Detection of coronary calcifications from computed tomography scans for automated risk assessment of coronary artery disease: Detection of coronary calcifications. Med Phys 2007; 34(4): 1450-61. Doi: 10.1118/1.2710548.
15. Kurkure U., Chittajallu D R., Brunner G., Le Y H., Kakadiaris I A. A supervised classification-based method for coronary calcium detection in non-contrast C T. Int J Cardiovasc Imaging 2010; 26(7): 817-28. Doi: 10.1007/s10554-010-9607-2.
16. de Vos B D., Wolterink J M., Leiner T., de Jong P A., Lessmann N., Isgum I. Direct Automatic Coronary Calcium Scoring in Cardiac and Chest C T. IEEE Trans Med Imaging 2019; 38(9): 2127-38. Doi: 10.1109/TMI.2019.2899534.
17. Martin S S., van Assen M., Rapaka S., et al. Evaluation of a Deep Learning-Based Automated C T Coronary Artery Calcium Scoring Algorithm. JACC Cardiovasc Imaging 2020; 13(2): 524-6. Doi: 10.1016/j.jcmg.2019.09.015.
18. Lee H., Martin S., Burt J R., et al. Machine Learning and Coronary Artery Calcium Scoring. Curr Cardiol Rep 2020; 22(9): 90. Doi: 10.1007/s11886-020-01337-7.
19. Greenland P., Blaha M J., Budoff M J., Erbel R., Watson K E. Coronary Calcium Score and Cardiovascular Risk. J Am Coll Cardiol 2018; 72(4): 434-47. Doi: 10.1016/j.jacc.2018.05.027.
20. Chang S., Kim H., Suh Y J., et al. Development of a deep learning-based algorithm for the automatic detection and quantification of aortic valve calcium. Eur J Radiol 2021; 137:109582. Doi: 10.1016/j.ejrad.2021.109582.
21. Rosenhek R., Binder T., Porenta G., et al. Predictors of Outcome in Severe, Asymptomatic Aortic Stenosis. N Engl J Med 2000:7.
22. Maeno Y., Abramowitz Y., Kawamori H., et al. A Highly Predictive Risk Model for Pacemaker Implantation After TAVR. JACC Cardiovasc Imaging 2017; 10(10): 1139-47. Doi: 10.1016/j.jcmg.2016.11.020.
23. Khalique O K., Hahn R T., Gada H., et al. Quantity and Location of Aortic Valve Complex Calcification Predicts Severity and Location of Paravalvular Regurgitation and Frequency of Post-Dilation After Balloon-Expandable Transcatheter Aortic Valve Replacement. JACC Cardiovasc Interv 2014; 7(8): 885-94. Doi: 10.1016/j.jcin.2014.03.007.
24. Pawade T, Clavel M A, Tribouilloy C, et al. Computed Tomography Aortic Valve Calcium Scoring in Patients With Aortic Stenosis. Circ: Cardiovascular Imaging. 2018; 11(3): e007146. doi: 10.1161/CIRCIMAGING. 117.007146

1-73. (canceled)

74. A computer-implemented method of locating an isotropic centroid of an aortic root comprising:
providing a non-contrast cardiac computed tomography image (CT image) to a multi-level convolutional neural network (ML-CNN);
processing the CT image using the ML-CNN, wherein the ML-CNN identifies 3D position and/or a 3D orientation of a plurality of anatomical features of the aortic root to predict a 3D position and/or a 3D orientation of the isotropic centroid of the aortic root on the CT image.

75. The method of claim 74, wherein the ML-CNN identifies the 3D position and a 3D orientation of the plurality of anatomical features of the aortic root to predict the 3D position and a 3D orientation of the isotropic centroid of the aortic root on the CT image.

76. The method of claim 75, wherein the ML-CNN predicts the 3D position and/or the 3D orientation of the isotropic centroid by performing the following steps:
a) processing/surveying the CT image to identify its 3D midpoint;
b) refining the spatial resolution and restricting the field of view to focus on the area surrounding the 3D midpoint of the CT image with an isotropic voxel spacing that captures the entire aortic root while keeping the image matrix constant;
c) identifying the 3D position and/or the 3D orientation of a plurality of anatomical features of the aortic root; and
d) predicting the 3D position and/or the 3D orientation of the isotropic centroid of the aortic root based on the 3D position and/or the 3D position of the anatomical features.

77. The method of claim 75, wherein steps (b) to (d) are repeated multiple times by the ML-CNN by further refining the spatial resolution and restricting the field of view each time while keeping the image matrix constant.

78. The method of claim 77, wherein the ML-CNN uses a previous prediction of the 3D position and/or the 3D orientation of the isotropic centroid of the aortic root as a starting point before predicting a new 3D position and/or 3D orientation of the isotropic centroid.

79. The method of claim 74, wherein the plurality of anatomical features are selected from the group consisting of an aortic valve annulus centroid, at least one aortic valve commissure, an aortic leaflet coaptation point, and at least one coronary ostia.

80. The method of claim 74, wherein the method additionally comprises:
training the ML-CNN to accurately identify 3D position and/or the 3D orientation of the plurality of anatomical features of the aortic root and to accurately predict the 3D position and/or the 3D orientation of the isotropic centroid of the aortic root based on the 3D position of the anatomical features.

81. A computer-implemented method of quantifying an amount of calcium in an aortic root comprising:
a) providing a non-contrast cardiac computed tomography image (CT image) to a deep-learning convolutional neural Network (DL-CNN), wherein a 3D position and/or a 3D orientation of an isotropic centroid of the aortic root on the CT image is known/has been identified previously;
b) processing the CT image to quantify the amount of calcium using the DL-CNN, wherein the DL-CNN:
i) segments a pre-determined region surrounding the isotropic centroid into a plurality of voxels;
ii) surveys each segmented voxel to ascertain whether it contains calcium; and
iii) calculates an Aortic Valve Calcification (AVC) score by quantifying the number of calcium containing segmented voxels that form part of the aortic root.

82. The method of claim 81, wherein the DL-CNN assigns a score of 1 to a segmented voxel that contains calcium and a score of 0 to a segmented voxel that does not contain calcium.

83. The method of claim 82, wherein the DL-CNN does not take into account a segmented voxel that contains calcium but does not form part of the aortic valve.

84. The method of claim 81, wherein the DL-CNN identifies the segmented voxel containing calcium by determining a CT attenuation value.

85. The method of claim 81, wherein the DL-CNN quantifies the amount of calcium of left cusp, right cusp, non-coronary cusp and left ventricular outflow tract [LVOT] separately.

86. The method of claim 81, wherein the DL-CNN is composed of more than one neural network models.

87. The method of claim 81, wherein the method additionally comprises:
training the DL-CNN to accurately quantify the amount of calcium in the aortic root; wherein the training comprises:
manually annotating a plurality of CT images and determining the amount of calcium in the aortic root to generate a training module; and
training the DL-CNN using the training module for a plurality of epochs.

88. The method of claim 87, wherein the training step further comprises:
providing structural parameters to the DL-CNN including a CT image pixel data, a binary mask of calcium based on thresholding, a 3D radial distance relative to the aortic root axis, and a projected out-of-plane 3D distance along the aortic root axis relative to the aortic root coaptation point.

89. The method of claim 88, wherein:
the CT pixel data is defined by μ∈ [symbol] ^{MNO} with an image matrix of dimension M×N×O, and positional indices i, j, k in the x, y, z directions respectively;
the binary mask is defined by t∈{0,1}^{MNO} of all calcium based on thresholding where:
$${t}_{i j k} = \{ \begin{matrix}0 & {μ}_{i j k} < 130 HU \\ 1 & {μ}_{i j k} \geq 130 HU\end{matrix} ;$$
the 3D radial distance relative to the aortic root axis r∈ [symbol] ^{MNO} where:
rᵢⱼₖ(v,\hat{n})=∥(xᵢⱼₖ−v)−\hat{n}((xᵢⱼₖ−v)·\hat{n})∥; and
the projected out-of-plane 3D distance is relative to the aortic root annular plane d∈ [symbol] ^{MNO} where:
dᵢⱼₖ(v,\hat{n})=(x_{i,j,k}−v)·\hat{n};
wherein;
xᵢⱼₖ is a spatial position at the center of voxel i, j, k, vis the annulus centroid position, \hat{n} is a unit normal vector of the aortic annulus plane.

90. The method of claim 89, wherein the DL-CNN provides a segmentation of the aortic root calcium mask (m) defined by
$$m = S ( μ , v , \hat{n} ) = {S}_{n n} ( [ \begin{matrix}μ \\ t ( μ ) \\ r ( v , \hat{n} ) \\ d ( v , \hat{n} )\end{matrix} ] )$$
using values of the structural parameters;
wherein m∈{0,1}^{MNO} is an array of the same shape as the CT images, wherein the DL-CNN assigns a score of 1 to a segmented voxel that contains calcium and a score of 0 to a segmented voxel that does not contain calcium.

91. A computer-implemented method of measuring/quantifying calcification of an aortic root using an algorithm comprising:
a) identifying a 3D position and/or a 3D orientation of a plurality of anatomical features of the aortic root on a non-contrast cardiac computed tomography image (CT image);
b) predicting a 3D position and/or a 3D orientation of the isotropic centroid of the aortic root on the CT image;
c) segmenting a pre-determined region surrounding the isotropic centroid into a plurality of voxels and surveying each segmented voxel to ascertain whether it contains calcium; and
d) calculating an Aortic Valve Calcification (AVC) score by quantifying the number of calcium containing segmented voxels that form part of the aortic root.

92. The method of claim 91, wherein the algorithm comprises a multi-level convolutional neural network (ML-CNN) and a deep learning convolutional neural network (DL-CNN).

93. The method of claim 91, wherein
steps a) and b) are carried out by providing a non-contrast cardiac computed tomography image (CT image) to a multi-level convolutional neural network (ML-CNN) and processing the CT image using the ML-CNN, wherein the ML-CNN identifies 3D position of a plurality of anatomical features of the aortic root to predict a 3D position of the isotropic centroid of the aortic root on the CT image; and
steps c) and d) are carried out by providing a non-contrast cardiac computed tomography image (CT image) to a deep-learning convolutional neural Network (DL-CNN), wherein a 3D position and/or a 3D orientation of an isotropic centroid of the aortic root on the CT image is known/has been identified previously, processing the CT image to quantify the amount of calcium using the DL-CNN, wherein the DL-CNN: i) segments a pre-determined region surrounding the isotropic centroid into a plurality of voxels, ii) surveys each segmented voxel to ascertain whether it contains calcium, and iii) calculates an Aortic Valve Calcification (AVC) score by quantifying the number of calcium containing segmented voxels that form part of the aortic root.

94. A non-transitory computer readable medium having stored thereon software instructions/an algorithm that, when executed by a processor, cause the processor to perform the method of claim 1.
